(19) United States
(12) Patent Application Publication
(10) Pub. No.: US 2025/0388539 A1
(43) Pub. Date: Dec. 25, 2025

(54) 3,3'-AROMATIC BIS(ETHER IMIDE)S, POLYETHERIMIDES THEREOF AND METHODS FOR MAKING

(71) Applicant: SHPP GLOBAL TECHNOLOGIES B.V., Bergen op Zoom (NL)

(72) Inventors: Satish Chandra PANDEY, Bangalore (IN); Juan J. RODRIGUEZ-ORDONEZ, Murcia (ES)

(21) Appl. No.: 18/880,385

(22) PCT Filed: Jun. 29, 2023
(86) PCT No.: PCT/IB2023/056767

§ 371 Date: Dec. 31, 2024

(30) Foreign Application Priority Data
Jul. 1, 2022 (EP) 22382630.6

(51) Int. Cl. C07D 209/48

(52) U.S. Cl. CPC C07D 209/48

(57) ABSTRACT

A method for the preparation of a 3-nitro-N—(C₁₋₁₃ alkyl)phthalimide composition comprises reacting a 3-nitro phthalic acid to provide 3-nitro-phthalic anhydride, optionally in the presence of a solvent, and reacting 3-nitro-phthalic anhydride with a C₁₋₁₃ alkylamine optionally in the presence of a solvent to provide the 3-nitro-N—(C₁₋₁₃ alkyl)phthalimide composition comprising 3-nitro-N—(C₁₋₁₃ alkyl)phthalimide and optionally, 4-nitro-N—(C₁₋₁₃ alkyl)phthalimide. The 3-nitro-N—(C₁₋₁₃ alkyl)phthalimide composition can have undetectable levels of 4-nitro-N—(C₁₋₁₃ alkyl)phthalimide, and consequently, polyetherimides ultimately derived from the 3-nitro-N—(C₁₋₁₃ alkyl)phthalimide composition can be enriched in 3,3′ linkages and/or exclude 3,4′ and 4′4 linkages. The polyetherimides disclosed can have improved flow and a decreased yellowness index.

### BACKGROUND

This disclosure relates to aromatic bis(ether imide)s, and in particular to 3,3′-aromatic bis(ether imide)s, polyetherimides thereof, method for making, and uses thereof.

Polyetherimides are a class of high performance polymers that can be processed to make molded articles, fibers, films, foams, and the like. Polyetheramides further have high strength, toughness, beat resistance, modulus, and broad chemical resistance, and so are widely used in industries as diverse as automotive, telecommunication, aerospace, electrical/electronics, transportation, and healthcare. Polyetherimides have shown versatility in various manufacturing processes, proving amenable to techniques including injection molding, extrusion, and thermoforming, to prepare the articles.

Polyetherimides can be prepared from aromatic bis(ether imide) monomers. Conventional methods for the preparation of aromatic bis(ether imide) monomers often result in isomeric mixtures including 3,3′-aromatic bis(ether imide), 3,4′-aromatic bis(ether imide), and 4,4′-aromatic bis(ether imide). The flow properties of polyetherimides derived from mixtures of aromatic bis(ether imide) isomers correlate to the ratio of 3,3′-aromatic bis(ether imide) to 3,4′-aromatic bis(ether imide) and 4,4′-aromatic bis(ether imide). Polyetherimides derived from aromatic bis(ether imide) isomer mixtures enriched in 3,4′-bis(ether imide) and 4,4′-aromnatic bis(ether imide) generally have lower flow than polyetherimides with a lesser amounts of 3,4′-aromatic bis(ether imide) and 4,4′-aromatic bis(ether imide) in the aromatic bis(ether imide) isomer mixture. Therefore, in applications where particular flow characteristics are desirable, there is a need for polyetherimides derived from monomer mixtures enriched in 3,3′-aromatic bis(ether imide). There accordingly remains a need in the art for methods for the preparation polyetherimides where the presence of 3,4′-aromatic bis(ether imide) and 4,4′-aromatic bis(ether imide) in the monomer mixture can be minimized, controlled to a pre-determined level, or eliminated.

### BRIEF DESCRIPTION

The above-described and other deficiencies of the art are met by a method for the preparation of a 3-nitro-N—(C₁₋₁₃ alkyl)phthalimide composition) comprising reacting 3-nitro phthalic acid optionally in the presence of a solvent, under conditions effective to provide a reaction mixture comprising 3-nitro-phthalic anhydride and water, and wherein the water is removed from the reaction mixture during the reacting, and combining 3-nitro-phthalic anhydride with a C₁₋₁₃ alkylamine optionally in the presence of a solvent under conditions effective to provide the 3-nitro-N—(C₁₋₁₃ alkyl)phthalimide composition comprising 3-nitro-N—(C₁₋₁₃ alkyl)phthalimide and optionally, 4-nitro-N—(C₁₋₁₃ alkyl)phthalimide.

A 3-nitro-N—(C₁₋₁₃ alkyl)phthalimide) composition comprises 3-nitro-N—(C₁₋₁₃ alkyl)phthalimide and optionally, 4-nitro-N—(C₁₋₁₃ alkyl)phthalimide, wherein the 3-nitro-N—(C₁₋₁₃ alkyl)phthalimide) composition comprises less than 20,000 ppm, less than 10,000 ppm, less than 5000 ppm, less than 2500 ppm or less than 1000 ppm of the 4-nitro-N—(C₁₋₁₃ alkyl)phthalimide.

A method for the preparation of an N—(C₁₋₁₃ alkyl)-3,3′-aromatic bis(ether imide) composition comprises reacting a dialkali metal salt of a dihydroxy aromatic compound with the 3-nitro-N—(C₁₋₁₃ alkyl)phthalimide composition prepared by the above method under conditions effective to form a product mixture comprising the N—(C₁₋₁₃ alkyl)-3,3′-aromatic bis(ether imide) composition comprising N—(C₁₋₁₃ alkyl)-3,3-aromatic bis(ether imide) and optionally, N—(C₁₋₁₃ alkyl)-3,4′-aromatic bis(ether imide). N—(C₁₋₁₃ alkyl)-4,4′-aromatic bis(ether imide), or a combination thereof, wherein when present, the N—(C₁₋₁₃ alkyl)-3,4′-aromatic bis(ether imide), the N—(C₁₋₁₃ alkyl)-4,4′-aromatic bis(ether imide), or a combination thereof.

A 3,3′-aromatic bis(ether imide) composition comprises 3,3′-aromatic bis(ether imide) and optionally, 3,4′-aromatic bis(ether imide), 4,4′-aromatic bis(ether imide), or a combination thereof, wherein the 3,3′-aromatic bis(ether imide) composition comprises less than 20,000 ppm, less than 10,000 ppm, less than 5000 ppm, less than 2500 ppm, or less than 1000 ppm of 3,4′-aromatic bis(ether imide), 4,4′-aromatic bis(ether imide), or a combination thereof.

A method for the manufacture of a polyetherimide comprises contacting the 3,3′-aromatic bis(ether imide) composition prepared by the above method with a phthalic anhydride in the presence of a catalyst and under conditions effective to provide a 3,3′-aromatic bis(ether phthaiic anhydride) composition comprising 3,3′-aromatic bis(ether phthalic anhydride) of formula (V-a) and optionally, 3,4′-aromatic bis(ether phthalic anhydride) of formula (V-b), 4,4′-aromatic bis(ether phthalic anhydride) of formula (V-c), or a combination thereof

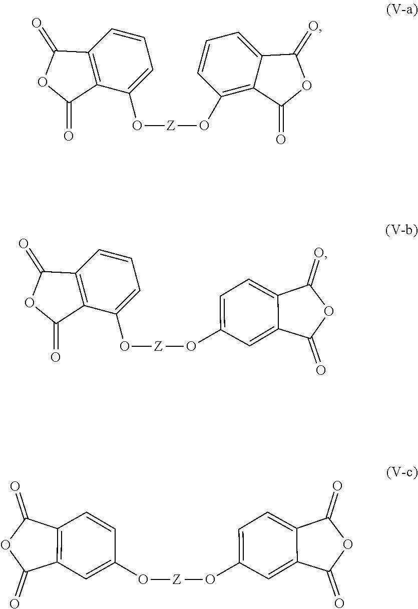

wherein Z is an aromatic C₆₋₂₄ monocyclic or polycyclic moiety optionally substituted with 1-6 C₁₋₈ alkyl groups, 1-8 halogen atoms, or a combination thereof; contacting the N—(C₁₋₁₃ alkyl)-3,3′-bis(ether phthalic anhydride) composition with an organic diamine of the formula (H₂N—R—NH₂) wherein R is a C₆₋₂₀ aromatic hydrocarbon group or a halogenated derivative thereof, a straight or branched chain C₂₋₂₀ alkylene group or a halogenated derivative thereof, a C₃₋₈ cycloalkylene group or halogenated derivative thereof, or a combination thereof.

Another method for the manufacture of a polyetherimide comprises hydrolyzing the 3,3′-aromatic bis(ether imide) composition prepared by the above method under conditions effective to provide the corresponding an aromatic bis(ether tetraacid) composition comprising an aromatic bis(ether tetracid) of formula (VII-a) and optionally, an aromatic bis(ether tetracid) of formula (VII-b), an aromatic bis(ether tetracid) of formula (VII-c), or a combination thereof

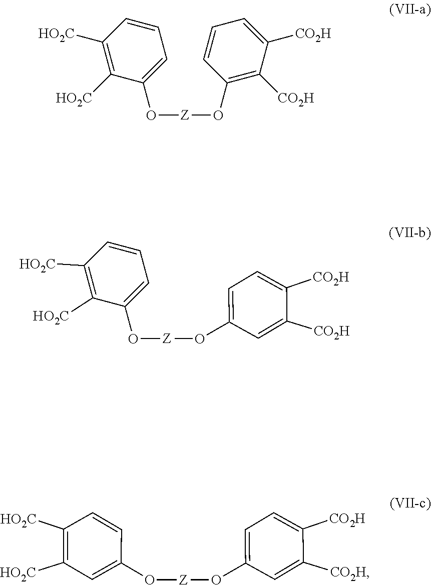

condensing the aromatic bis(ether tetraacid) composition under conditions effective to provide a an aromatic bis(ether phthalic anhydride) composition comprising 3,3′-aromatic bis(ether phthaiic anhydride) and optionally, a 3,4′-aromatic bis(ether phthaiic anhydride), 4,4′-aromatic bis(ether phthalic anhydride), or a combination thereof,

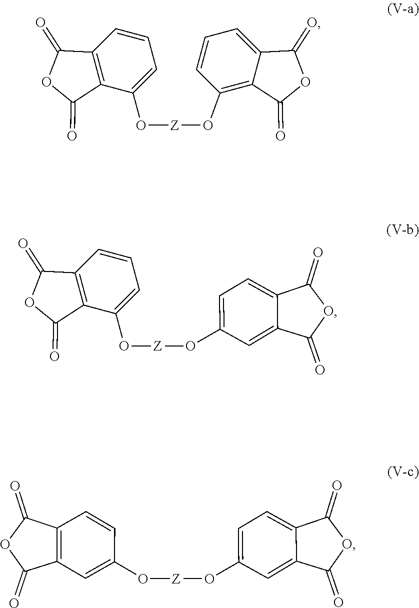

wherein Z is an aromatic C₆₋₂₄ monocyclic or polycyclic moiety optionally substituted with 1-6 C₁₋₈ alkyl groups, or 1-8 halogen atoms; and contacting the 3,3′-aromatic bis(ether phthalic anhydride) composition with an organic diamine of the formula (H₂N—R—NH₂) wherein R is a C₆₋₂₀ aromatic hydrocarbon group or a halogenated derivative thereof, a straight or branched chain C₂₋₂₀ alkylene group or a halogenated derivative thereof, or a C₃₋₈ cycloalkylene group or halogenated derivative thereof.

A polyetherimide comprises repeating units of formula (VIII-a) and optionally, repeating units of formula (VIII-b), repeating units of formula (VIII-c), or a combination thereof

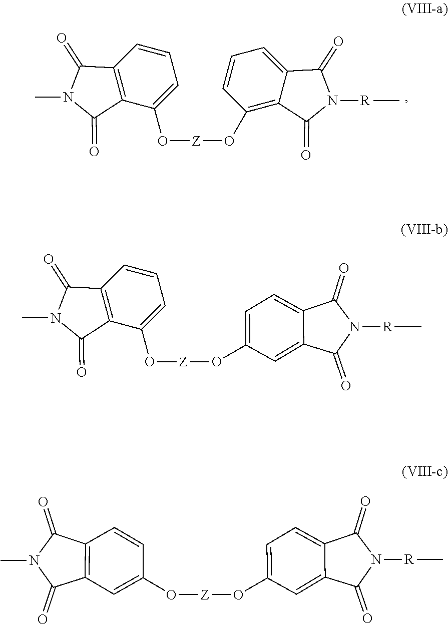

wherein Z is an aromatic C₆₋₂₄ monocyclic or polycyclic moiety optionally substituted with 1-6 C₁₋₈ alkyl groups, 1-8 halogen atoms, or a combination thereof, R is a C₆₋₂₀ aromatic hydrocarbon group or a halogenated derivative thereof, a straight or branched chain C₂₋₂₀ alkylene group or a halogenated derivative thereof, or a C₃₋₈ cycloalkylene group or halogenated derivative thereof, and wherein the polyetherimide comprises less than 20.000 ppm, less than 10,000 ppm, less than 5000 ppm, less than 2500 ppm, or less than 1000 ppm of repeating units of formula (VIII-b), repeating units of formula (VIII-c, or a combination thereof.

An article comprises the above described polyetherimide.

A method of manufacturing the above article is disclosed.

The above described and other features are exemplified by the following detailed description, examples, and claims.

### DETAILED DESCRIPTION

Conventional methods for the preparation of aromatic bis(ether imide) monomers often result in isomeric mixtures of 3,3′-aromatic bis(ether imide), 3,4′-aromatic bis(ether imide), and 4,4′-aromatic bis(ether imide). For example, conventional methods for preparing aromatic bis(ether imide) use chlorophthalic anhydride, which is a mixture of 4-chlorophthalic anhydride and 3-chlorophthalic anhydride in about a 95:5 ratio as obtained from suppliers. Although the isomers are separable by distillation, the boiling points are very close (i.e., 290° C. for 4-chlorophthalic anhydride and 295° C. for 3-chlorophthalic anhydride, each at atmospheric pressure), so a certain amount of 4-chlorophthalic anhydride is present in the 3-chlorophthalic anhydride distillate and a certain amount of 3-chlorophthalic anhydride is present in the 4-chlorophthalic anhydride distillate. As a result, there is some loss of the 3-chlorophthalic anhydride to the 4-chlorophthalic anhydride distillate and the 4-chlorophthalic anhydride that co-distilled with the 3-chlorophthalic anhydride is commonly carried through the synthesis, ultimately resulting in a mixture aromatic bis(ether imide) isomers including 3,3′-aromnatic bis(ether imide), 3,4′-aromatic bis (ether imide), and 4-4′-aromatic bis(ether imide) (shown below as derived from bisphenol A for illustrative purposes only).

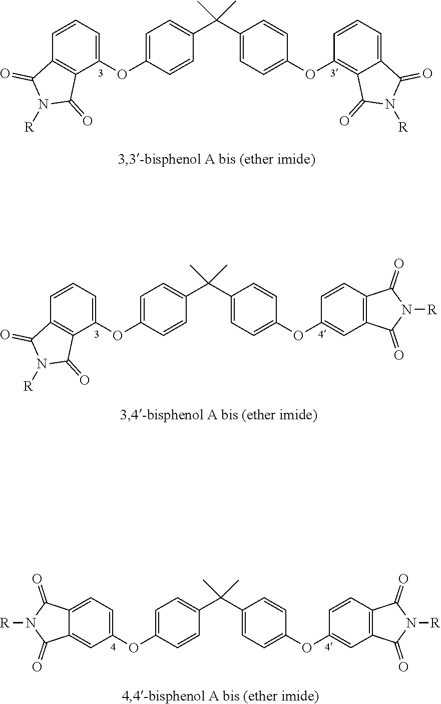

The inventors have discovered methods for preparing 3,3′-aromatic bis(ether imide) from 3-nitro phthalic acid that can minimize or eliminate the formation of 3,4′-aromatic bis(ether imide) and 4,4′-aromatic bis(ether imide). As a result, polyetherimides derived from the aromatic 3,3′-bis(ether imide) composition include very low levels or no repeating units derived from 3,4′-aromatic bis(ether imide) and 4,4′-aromatic bis(ether imide). As previously discussed, polyetherimides derived from a monomer mixture enriched in 3,3′-aromatic bis(ether imide) have improved flow, which is desirable for applications where higher flow is advantageous. As an added advantage, the yellowness index (YI) of the 3,3′-aromatic bis(ether imide)-rich monomer mixtures and the polyetherimides derived from 3,3′-aromatic bis(ether imide)-rich monomer mixtures may be lower than for aromatic bis(ether imide)s and polyetherimides prepared using conventional methods.

The disclosed methods are also an improvement over other conventional methods that use nitric acid to introduce the nitro substituent onto the aromatic ring of an N-alkyl phthalimide, which results in a mixture of 3-nitro-N-alkyl phthalimide, 4-nitro-N-alkyl phthalimide, and 4-hydroxy-3,5-dinitro-N-alkylphthaimide, wherein 4-nitro-N-alkyl phthalimide is the major product.

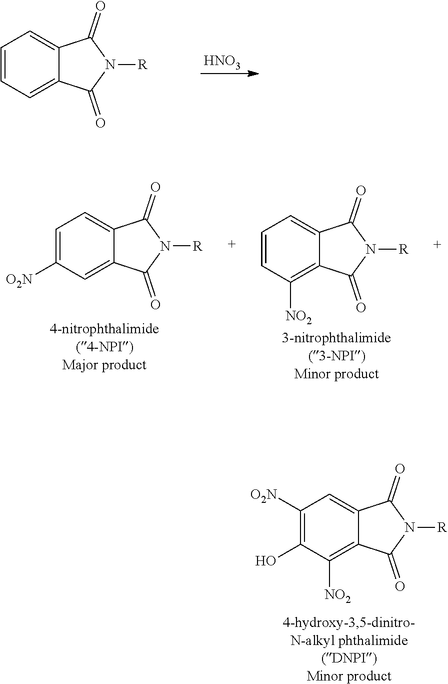

As shown above, a mixture of 3-nitro-N-alkyl phthalimide and 4-nitro-N-alkyl phthalimide is obtained during nitration, and ultimately any polyetherimide derived from such a mixture can have a higher level of repeating units derived from 3,4′-aromatic bis(ether imide) and 4,4′-aromatic bis(ether imide), thus resulting in a lower flow and a higher YI. In addition, this conventional method wherein N-alkyl phthalimide is nitrated results in the formation of the 3,5-dinitro-4-hydroxyphthalimide, which lowers the overall yield of the desired products and must be removed. Therefore, the disclosed methods are an improvement over this conventional method because 3-nitro-N-alkyl phthalimide can be prepared as the major product, uncontaminated with significant amounts of 4-nitro-N-alkyl phthalimide, and can exclude 4-nitro-N-alkyl phthalimide. The disclosed methods avoid the formation of 3,5-dinitro-4-hydroxyphthalimide as well.

As an added advantage, the disclosed methods avoid the use of halogenated synthetic intermediates, enabling the preparation of monomers and polyetherimides with reduced halogen content. In some aspects, the monomers and polyetherimides can be essentially halogen-free as well as having an improved flow and YI. As used herein, the phrase “essentially halogen-free” is as defined by IEC 61249-2-21 or UL 746H. According to International Electrochemical Commission, Restriction Use of Halogen (IEC 61249-2-21), a composition should include 900 parts per million (ppm) or less of each of chlorine and bromine and also include 1500 ppm or less of total bromine, chlorine, and fluorine content. According to UL 746H, a composition should include 900 ppm or less of each of chlorine, bromine, and fluorine and 1500 ppm or less of the total chlorine, bromine, and fluorine content. The bromine, chlorine, and fluorine content in ppm may be calculated from the composition or measured by elemental analysis techniques.

Accordingly, another aspect of the present disclosure is a method for producing an N-alkyl phthalimide composition. The method comprises first reacting 3-nitrophthalic acid to form a 3-nitrophthalic anhydride composition. The 3-nitro phthalic acid is essentially free of 4-nitrophthalic acid, resulting in 3-nitrophthalic anhydride essentially free of 4-nitrophthalic anhydride, a 3-nitro-N-alkyl phthalimide composition essentially free of 4-nitro-N-alkyl phthalimide, a 3,3′-aromatic bis(ether imide) composition essentially free of either 3,4′-aromatic bis(ether imide) or 4,4′-aromatic bis(ether imide), and polyetherimides essentially free of 3,4′ or 4,4′ linkages in the chain. As used herein the term “essentially free” means that the presence of the component is undetectable by analytical methods, such as NMR, LC-MC, HPLC, GC-MS, and the like.

In some aspects, 3-nitrophthalic acid may contain a very low level of 4-nitrophthalic acid. Indeed, the isomeric purity of the 3-nitro-phthalic acid starting material is related to the isomeric purity of the downstream synthetic intermediates and polyetherimide. One of ordinary skill in the art would understand that the purity of 3-nitrophthalic acid may vary with the supplier and may contain a very low level (i.e., ppm levels) of 4-nitrophthalic acid and that such low levels of 4-nitrophthalic acid may ultimately provide polyetherimides having the desired flow properties even though the polyetherimides include a limited amount of 3,4′- and 4,4′-linkages in the polymeric chain.

The cyclization of 3-nitrophthalic acid can be accomplished with heating, optionally in the presence of a solvent. The conversion of 3-nitrophthalic acid to 3-nitrophthalic anhydride can be performed in the absence of solvent by heating the 3-nitrophthalic acid so that the 3-nitrophthalic acid begins to melt. The 3-nitrophthalic acid can be partially melted or completely melted. As the anhydride is formed, water is produced by the reaction mixture, which is removed from the reaction mixture as the reaction proceeds. When the reaction is complete or near completion, the water production slows or stops.

In a preferred aspect, the 3-nitrophthalic anhydride is prepared in the absence of solvent. Advantageously, under solvent-free conditions, the conversion of 3-nitrophthalic acid to 3-nitrophthalic anhydride is completed after about 1 hour. This is a much shorter reaction time than the conversion of 4-chlorophthalic acid to 4-chlorophthalic anhydride, with reaction times ranging from 6-10 h when water is efficiently removed from the reactor. If solvent is used, the solvent can either be removed or the reaction mixture including the 3-nitrophthalic anhydride and solvent can be carried on to the next step without isolating the 3-nitrophthalic anhydride. The solvent-free approach is preferred as it avoids the use of solvents, which are an added expense and the reaction proceeds at lower temperatures, therefore decreasing energy usage for this step.

The method for producing a 3-nitro-N—(C₁₋₃ alkyl) phthalimide composition includes reacting the 3-nitrophthalic anhydride with a C₁₋₁₃ alkylamine. This reaction can be performed with heating and optionally in the presence of a solvent. The conversion of 3-nitrophthalic anhydride to nitro-N—(C₁₋₁₃ alkyl)phthalimide can be performed in the absence of solvent by heating the 3-nitrophthalic acid so that the 3-nitrophthalic acid begins to melt. The 3-nitrophthalic acid can be partially melted or completely melted. Similar to the previous step, as the 3-nitro-N—(C₁₋₁₃ alkyl) phthalimide is formed, water is produced by the reaction mixture, which is removed from the reaction mixture as the reaction proceeds. The water production stops when the reaction is complete. Preferably, the 3-nitro-N—(C₁₋₁₃ alkyl) phthalimide is essentially free of 4-nitro-N—(C₁₋₁₃ alkyl) phthalimide. As used herein “essentially free of 4-nitro-N—(C₁₋₁₃ alkyl) phthalimide” means that the presence of 4-nitro-N—(C₁₋₁₃ alkyl) phthalimide is not detectable in the 3-nitro-N-alkyl phthalimide composition by analytic methods (e.g., LC-MS. HPLC, GC-MS). Suitable HPLC conditions may be found in U.S. Pat. No. 4,902,809.

Depending on the purity of the 3-phthalic acid, the 3-nitro-N—(C₁₋₁₃ alkyl)phthalimide composition can include low amounts of 3-nitro-N—(C₁₋₁₃ alkyl)phthalimide, such as, for example, less than 20,000 ppm, less than 10,000 ppm, less than 5000 ppm, less than 2500 ppm, less than 1000 ppm, less than 500 ppm, or less than 100 ppm of 4-nitro-N—(C₁₋₁₃ alkyl)phthalimide. In some aspects, the presence of 4-nitro-N-alkyl phthalimide is not detectable in the 3-nitro-N-alkyl phthalimide composition by analytic methods (e.g., LC-MS, 1PLC, GC-MS). Suitable 1-PLC conditions may be found in U.S. Pat. No. 4,902,809. When the presence of 4-nitro-N—(C₁₋₁₃ alkyl)phthalimide is undetectable by analytic methods, the, N—(C₁₋₁₃ alky)phthalimide is essentially free of 4-nitro-N—(C₁₋₁₃ alkyl)phthalimide.

The anhydride formation and phthalimide formation can be carried out at a temperature of less than or equal to 250° C., for example about 150-250° C., or 150-225° C. Temperatures outside the range of temperatures disclosed above also can be used; however, lower temperatures can result in a reaction rate that is too slow to be cost effective.

The pressure range under which the nitration process can vary from vacuum to above atmospheric pressure. Such conditions, however, depend on the type of reactor or reactors employed. Otherwise, the process is generally run at atmospheric pressure.

The yield from the conversion of 3-nitrophthalic acid to the 3-nitro-N—(C₁₋₁₃ alkyl)phthalimide composition may be improved. The % yield may be at least 60%, or 65%, or 70%, or 75%, or 80%, based on the weight of 3-nitrophthalic acid.

Accordingly, another aspect of the present disclosure is a method for producing an aromatic bis(ether imide) monomer. The method comprises reacting a dialkali metal salt of a dihydroxy aromatic compound with the nitro-N-alkyl phthalimide composition under conditions effective to form a product mixture comprising the aromatic bis(ether imide) monomer.

The particular conditions for reacting the dialkali metal salt of a dihydroxy aromatic compound with the 3-nitro-N-alkyl phthalimide composition to provide the aromatic bis(ether imide) will depend on the specific dihydroxy aromatic compounds, the specific components of the nitro-N-alkyl phthalimide composition, the solvent, the presence of or absence of a phase transfer catalyst, and like considerations. For example, the reacting can be at a temperature of about 25-250° C., for example, 100-250° C., or 115-200° C., or 100-125° C., or 115-125° C. The reacting can be at atmospheric pressure, super-atmospheric pressure, or sub-atmospheric pressure. For example, the reacting can be at a pressure of 0-70 kPa, or 30-70 kPa, or 50-70 kPa, or 10-30 kPa, or 10-40 kPa, or 10-50 kPa, or 10-60 kPa, or 20-40 kPa, or 20-50 kPa, or 20-60 kPa, or 30-50 kPa, or 30-60 kPa, or 40-60 kPa.

The reaction mixture can have a solids content of 1-90 wt %, or 10-90 wt %, or 10-80 wt %, or 10-70 wt %, or 10-60 wt %, or 40-90 wt %, or 50-90 wt %, or 60-90 wt %, or 10-50 wt %, or 20-50 wt %, or 30-50 wt %, or 10-40 wt %, or 10-30 wt %, or 20-40 wt %, each based on the total weight of the reaction mixture, depending on the nature of the N-alkyl group. In some aspects, the reaction mixture can have a solids content of 20-30 wt %, or 22-26 wt % based on the total weight of the reaction mixture. As used herein, “solids content” refers to the weight of the non-solvent components whether dissolved or in solid form divided by the total weight of the reaction mixture.

One mole equivalent of dialkali metal salt and 2 mole equivalents of nitro-N-alkyl phthalimide composition can be used, while higher or lower amounts of either will not substantially interfere with the formation of the desired aromatic bis(ether imide). In some aspects, however, two moles of the nitro-N-alkyl phthalimide composition per mole of dialkali metal salt is preferred. In some aspects, the molar ratio of dialkali metal salt to the nitro-N-alkyl phthalimide composition can be 1:1.5 to 1:2.5, or 1:1.7 to 1:2.3, or 1:1.8 to 1:2.2, or 1:1.9 to 1:2.1.

In some aspects, the reaction to prepare the aromatic bis(ether imide) is conducted in the presence of a solvent. Any organic solvent which does not react with the reactants during the formation of the aromatic bis(ether imide) can be used in the reaction. In some aspects, the solvent comprises a nonpolar organic solvent. Suitable nonpolar organic solvents include, but are not limited to, toluene, benzene, chlorobenzene, bromobenzene, dichlorobenzenes (e.g., ortho-, meta-, or para-dichlorobenzene), trichlorobenzenes (e.g., 1,2,4-trichlorobenzene), xylene (including m-xylene, o-xylene, p-xylene, and combinations comprising at least one of the foregoing), anisole, ethylbenzene, propylbenzene, mesitylene, and the like, or a combination thereof. In some aspects, the solvent can be toluene, benzene, chlorobenzene, ortho-dichlorobenzene, 1,2,4-trichlorobenzene, xylene, and the like, or a combination thereof nonpolar organic solvents. In some aspects, the solvent preferably comprises toluene.

The solvent can comprise a dipolar aprotic solvent. Suitable dipolar aprotic solvents can include, but are not limited to, dimethyl sulfoxide, dimethylacetamide, N-methylpyrrolidone, 1-cyclohexyl-2-pyrrolidone, N-isopropyl-pyrrolidone, tetramethylurea, dimethylformamide, sulfolane, N-methylcaprolactam, and the like, or a combination thereof dipolar aprotic solvents. In some aspects, the solvent can be a combination of a nonpolar organic solvent and a dipolar aprotic solvent. For example, a nonpolar organic solvent and a dipolar aprotic solvent can be present in a weight ratio of 1:99 to 99:1, or 5:95 to 95:5, or 10:90 to 90:10, or 20:80 to 80:20, or 30:70 to 70:30, or 40:60 to 60:40.

The solids content of the product mixture comprising the aromatic bis(ether imide) can be 5-90 wt %, or 10-90 wt %, or 10-80 wt %, or 10-70 wt %, or 10-60 wt %, or 10-50 wt %, or 10-40 wt %, or 10-30 wt %, or 10-20 wt %, or 5-80 wt %, or 5-70 wt %, or 5-60 wt %, or 5-50 wt %, or 5-40 wt %, or 5-30 wt % or 5-20 wt %; or 10-90 wt %, or 10-80 wt %, or 10-70 wt %, or 10-60 wt %, or 10-50 wt %, or 10-40 wt %, or 10-30 wt %, or 10-20 wt %, or 20-90 wt %, or 20-80 wt %, or 20-70 wt %, or 20-60 wt %, or 20-50 wt %, or 20-40 wt %, or 20-30 wt %.

In some aspects, the reacting can be in the presence of a phase transfer catalyst. A wide variety of phase transfer catalysts can be used, for example various phosphonium, ammonium, guanidinium, and pyridinium salts can be used. The phase transfer catalyst can be a hexa(C₁₋₁₂ alkyl)guanidinium salt, a tetra(C₁₋₁₂ alkyl)ammonium salt, a tetra(C₁₋₁₂alkyl) phosphonium salt, or a tetra(C₆₋₂₀ aryl) phosphonium salt. For example, the phase transfer can be tetraethylammonium bromide, tetraethylammonium acetate, tetrabutylammonium bromide, tetrapropylammonium bromide, tetrabutylammonium chloride, tetrabutylammonium fluoride, tetrabutylammonium acetate, tetrahexylammonium chloride, tetraheptylammonium chloride. Aliquat 336 phase transfer catalyst, tetrabutylphosphonium bromide, tetraphenylphosphonium bromide, tetrabutylphosphonium chloride, hexaethylguanidinium chloride, and the like. A pyridinium salt, for example a bis-aminopyridinium salt can also be used.

The phase transfer catalyst can be a quaternary salt or a bis-quaternary salt. Among the quaternary salts that can be used are catalysts of the formula (R³)₄Q⁺X, wherein each R³ is the same or different, and is a C₁₋₁₀ alkyl; Q is a nitrogen or phosphorus atom; and X is a halogen atom or a C₁₋₈ alkoxy or C₆₋₁₈ aryloxy. Exemplary phase transfer catalysts include (CH₃(CH₂)₃)₄NX, (CH₃(CH₂)₃)₄PX, (CH₃(CH₂)₅)₄NX, (CH₃(CH₂)₆)₄NX, (CH₃(CH₂)₄)₄NX, CH₃(CH₃(CH₂)₃)₃NX, and CH₃(CH₃(CH₂)₂)₃NX, wherein X is Cl⁻, Br⁻, a C₁₋₈ alkoxy or a C₆₋₁₈ aryloxy.

Among the bis-quaternary salts that can be used are those of the formula (R)ₖQ⁺(R³)ₘQ(R⁴)ₖ (X²)₂ wherein each R³ is independently a divalent C₁₋₆₀ hydrocarbon group, all R³ taken together contain 4-54 carbon atoms, each R⁴ is independently a C₁₋₁₂ hydrocarbon group, Q is nitrogen or phosphorus, preferably nitrogen, X² is an organic or inorganic anionic atom or group, k is an integer from 1-3, and rm is 4-k, wherein at least three of R³ and R⁴ groups attached to each Q atom are aliphatic or alicyclic. In particular, each R³ can be a divalent C₁₋₁₈ alkylene, C₃₋₈ cycloalkylene, or C₆₋₁₈ aromatic group such as ethylene, propylene, trimethylene, tetramethylene, hexamethylene, octamethylene, decamethylene, dodecamethylene, cyclohexylene, phenylene, tolylene, or naphthylene, or a C₃₋₁₂ divalent heterocyclic group derived from a compound such as pyridine or indole. In some aspects, each R³ is C₁₋₁₂ alkylene, specifically C₃₋₈ alkylene. Preferably, only one R³ group is present (i.e., m is 1 and each k is 3) and it contains 5-10, specifically 6 carbon atoms. Illustrative R⁴ groups are methyl, ethyl, n-propyl, isopropyl, n-butyl, isobutyl, n-hexyl, n-heptyl, cyclopentyl, cyclohexyl, methylcyclohexyl, phenyl, tolyl, 2-(1,4-dioxanyl) and 2-furyl. Preferably, the R⁴ groups are all alkyl, for example C₁₋₄ n-alkyl groups. The X² can be any anion that is stable under the conditions used; suitable anions include chloride, bromide, sulfate, p-toluenesulfonate, and methanesulfonate, preferably bromide. The value of the integer k can be from 1-3, and the value of m is 4-k. In some aspects, each k is 3 and m is 1. In some aspects, all of the R³ and R⁴ groups are aliphatic. Illustrative bis-quaternary salts of this type include those in which R³ is a polymethylene chain from trimethylene to dodecamethylene, each R⁴ is either n-butyl or n-hexyl, Q is nitrogen. X² is bromide, each k is 2 and m is 2; the compound in which each R³ is ethylene, R⁴ is n-butyl, Q is nitrogen, X² is bromide, each k is 1 and in is 3; and the compound in which R³ is hexamethylene, each R⁴ is n-butyl, Q is phosphorus, X² is bromide, each k is 3 and m is 1.

Quaternary salts that can be used as phase transfer catalysts include quaternary salts of dihydroxy aromatic compounds as described in U.S. Pat. No. 5,756,843 to Webb et al. For example, a quaternary salt of a dihydroxy aromatic compound can be of the formula A⁺(O—Z—O)₂H₃, wherein A is a monocationic carbon- and nitrogen- or phosphorus containing group (i.e., a group having a single positive charge comprising carbon and nitrogen or carbon and phosphorus). The group A comprises 1-6 C₂₋₁₂alkyl groups. In some aspects, A preferably comprises nitrogen. In some aspects, A can be a tetra(C₂₋₁₂alkyl)ammonium or tetra(C₂₋₁₂alkyl)phosphonium group, for example tetraethylammonium, tetra-n-butylammonium, tetra-n-butylphosphonium and diethyl di-n-butylammonium. In some aspect, A is preferably a hexa(C₂₋₁₂alkyl)guanidinium group, for example hexaethylguanidinium, hexa-n-butylguanidinium, or tetraethyldi-n-butylguanidinium. Z is an aromatic C₆₋₂₄ monocyclic or polycyclic moiety optionally substituted with 1-6 C₁₋₈ alkyl groups, 1-8 halogen atoms, or a combination thereof. In some aspects, Z is of formula (IIa) as described below. Z is 2,2-(4-phenylene)isopropylidene (i.e., the dihydroxy aromatic compound from which Z is derived is 2,2-bis-(4-hydroxyphenyl)propane or bisphenol A). The quaternary salts of dihydroxy aromatic compounds can be prepared, for example, by the reaction of a dihydroxyaromatic compound of the formula HO—Z—OH with an alkali metal hydroxide and a quaternary salt of the formula A⁺X. The group X can be as described above, and is a halide, or bromide or chloride and most preferably chloride. Typical reaction temperatures are about 1-125° C., preferably about 10-50° C., and preferably under an inert atmosphere such as nitrogen or argon.

In some aspects, the phase transfer catalyst is preferably a hexa(C₁₋₁₂alkyl)guanidinium salt, for example hexaethylguanidinium chloride.

The phase transfer catalyst can be present in an amount of 0.1-10 mole percent (mol %), 0.5-10 mol %, 0.5-5.0 mol %, based on the total moles of the dialkali metal salt of the dihydroxy aromatic compound. In some aspects, the phase transfer catalyst can be present in an amount of 0.1-2.5 mol %, or 0.5-2.5 mol %. It has been found that in the disclosed methods, the amount of catalyst needed can be less than conventional approaches wherein 4-hydroxy-3,5-dinitro-N—(C₁₋₁₃ alkyl)phthalimide (DNPI) is formed as a side-product.

The aromatic bis(ether imide) can be recovered from the product mixture and purified by a variety of procedures. One procedure includes dissolution of the aromatic bis(ether imide) in an organic solvent such as toluene and then washing or extracting with alkali solution containing 0.1-10 wt %, or 1-5 wt % alkali, to remove by-products, e.g., monoimides, and the like, phase transfer catalyst, and unreacted starting materials. In some aspects, the volumetric ratio of the alkali solution to the organic phase (e.g., the aromatic bis(ether imide) in organic solvent) during the washing or extracting can be 1:5 to 1:15, or 1:5 to 1:10, or 1:6 to 1:9, or 1:6 to 1:8.

The aromatic bis(ether imide)s prepared according to the above method can be obtained in a yield of greater than 75%, greater than 80%, or greater than 85%, or greater than 90%.

After recovery, the aromatic bis(ether imide) can be of high or low color, as indicated by yellowness index (YI). YI is a value calculated from spectrophotometric data that describes the color of a test sample as being clear or white (low YI) versus being more yellow (high YI). Sample handling and preparation can affect the test results. YI of the aromatic bis(ether imide) can be measured according to ASTM D1925, by dissolving 0.5 g of aromatic bis(ether imide) in 10 milliliters of methylene chloride, and measuring the YI of the resulting solution, for example on an Xrite 7000 Color Eye device (Xrite, Incorporated). In some aspects, the YI of the aromatic bis(ether imide) can be 15 or less, or 10 or less, for example 1-15 or 1-10. In a preferred aspect, the YI of the aromatic bis(ether imide) can be 5 or less, determined in accordance with ASTM D-1925 at a thickness of 3.2 mm. For example, the aromatic bis(ether imide) can have a YI of 1-15, or 1-10, 5-15, or 5-10, or 1-9, or 1-8, or 1-7, or 1-6, or 1-5, or 1-4, or 1-3, or 2-9, or 2-8, or 2-7, or 2-6, or 2-5, or 2-4, or 3-9, or 3-8, or 3-7, or 3-6, or 3-5, or 4-9, or 4-8, or 4-7, or 4-6, or 5-9, or 5-8, or 5-7, or 6-9, or 6-8, or 7-9, determined in accordance with ASTM D-1925.

In an aspect, the method for producing an aromatic bis(ether imide) preferably comprises reacting a dialkali metal salt of a dihydroxy aromatic compound with a nitro-N-alkyl phthalimide composition in the presence of a hexaethylguanidinium chloride phase transfer catalyst and under conditions effective to form a product mixture comprising the aromatic bis(ether imide), wherein the nitro-N-alkyl phthalimide composition comprises 3-nitro-N—(C₁₋₁₃ alkyl)phthalimide and optionally, 4-nitro-N—(C₁₋₁₃ alkyl)phthalimide and the aromatic bis(ether imide) has a YI of less than 15, or less than 10, or less than 5, as determined according to ASTM D-1925.

In some aspects, the aromatic bis(ether imide) composition before isolation of the aromatic bis(ether imide) composition from the reaction mixture and after isolation of the aromatic bis(ether imide) composition from the reaction mixture can be essentially free of 4-hydroxy-3,5-dinitro-N—(C₁₋₁₃ alkyl)phthalimide, which means that the presence of 4-hydroxy-3,5-dinitro-N—(C₁₋₁₃ alkyl)phthalimide is not detectable, for example by a high performance liquid chromatography (HPLC) method after recovery. Suitable HPLC conditions may be found in U.S. Pat. No. 4,902,809.

The dialkali metal salt of the dihydroxy aromatic compound is of formula (I)

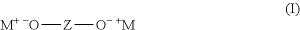

wherein M is an alkali metal and Z is an aromatic C₆₋₂₄ monocyclic or polycyclic moiety optionally substituted with 1-6 C₁₋₈ alkyl groups, 1-8 halogen atoms, or a combination thereof. The alkali metal M can be, for example, lithium, sodium, potassium, or a combination thereof. In some aspects, M is sodium. Exemplary groups Z include groups derived from an aromatic dihydroxy compound of formula (II)

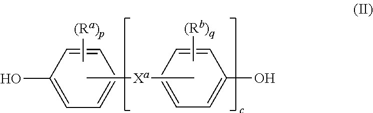

wherein R^{a} and R^{b} can be the same or different and are a halogen atom or a monovalent C₁₋₆ alkyl group, for example; p and q are each independently integers of 0-4; c is 0-4; and X^{a} is a bridging group connecting the hydroxy-substituted aromatic groups, where the bridging group and the hydroxy substituent of each C₆ arylene group are disposed ortho, meta, or para (specifically para) to each other on the C₆ arylene group. The bridging group X^{a} can be a single bond. —O—, —S—, —S(O)—, —S(O)₂—, —C(O)—, or a C₁₋₁₈ organic bridging group. The C₁₋₁₈ organic bridging group can be cyclic or acyclic, aromatic or non-aromatic, and can further comprise heteroatoms such as halogens, oxygen, nitrogen, sulfur, silicon, or phosphorous. The C₁₋₁₈ organic group can be disposed such that the C₆ arylene groups connected thereto are each connected to a common alkylidene carbon or to different carbons of the C₁₋₁₈ organic bridging group. A specific example of a group Z is a divalent group of formula (II-a)

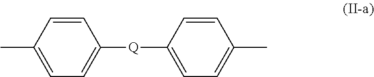

wherein Q is —O—, —S—, —C(O)—, —SO₂—, —SO—, or —C_{y}H_{2y}— wherein y is an integer from 1-5 or a halogenated derivative thereof. Exemplary dihydroxy aromatic compounds from which Z can be derived include but are not limited to 2,2-bis(2-hydroxyphenyl)propane, 2,4′-dihydroxydiphenylmethane, bis(2-hydroxyphenyl)methane, 2,2-bis-(4-hydroxyphenyl)propane (“bisphenol A” or “BPA”), 11-bis-(4-hydroxyphenyl)ethane, 1,1-bis-(4-hydroxyphenyl)propane, 2,2-bis-(4-hydroxyphenyl)pentane, 3,3′-bis-(4-hydroxyphenyl)pentane, 4,4′-dihydroxybiphenyl, 4,4′-dihydroxy-3,3,5,5′-tetramethylbiphenyl, 2,4′-dihydroxybenzophenone, 4,4′-dihydroxydiphenylsulfone, 2,4′-dihydroxydiphenylsulfone, 4,4′-dihydroxydiphenylsulfoxide, 4,4′-dihydroxydiphenylsulfide, hydroquinone, resorcinol, 3,4-dihydroxydiphenylmethane, 4,4-dihydroxybenzophenone, 4,4′-dihydroxydiphenylether, and the like, or a combination thereof. In some aspects, Z is 2,2-(4-phenylene)isopropylidene (i.e. the dihydroxy aromatic compound from which the dialkali metal salt is derived is 2,2-bis-(4-hydroxyphenyl)propane or bisphenol A, such that Q in formula (IIa) is 2,2-isopropylidene).

The nitro-N-alkyl phthalimide composition comprises a 3-nitro-N—(C₁₋₃ alkyl)phthalimide of formula (III-a) and optionally, a 4-nitro-N—(C₁₋₁₃ alkyl) phthalimide of formula (III-b)

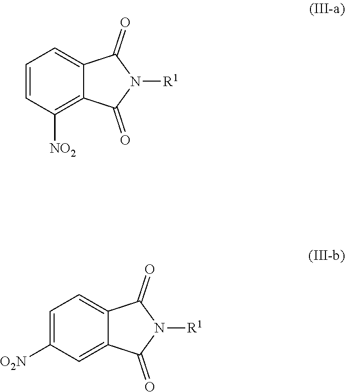

wherein R¹ is a monovalent C₁₋₁₃ alkyl group, preferably a C₁₋₄ alkyl group, for example a methyl group.

The 3,3′-aromatic bis(ether imide) composition includes a 3,3′-aromatic bis(ether imide) of formula (IV-a) and optionally, a 3,4′-aromatic bis(ether imide) of formula (IV-b), a 4,4′-aromatic bis(ether imide) of formula (IV-c)

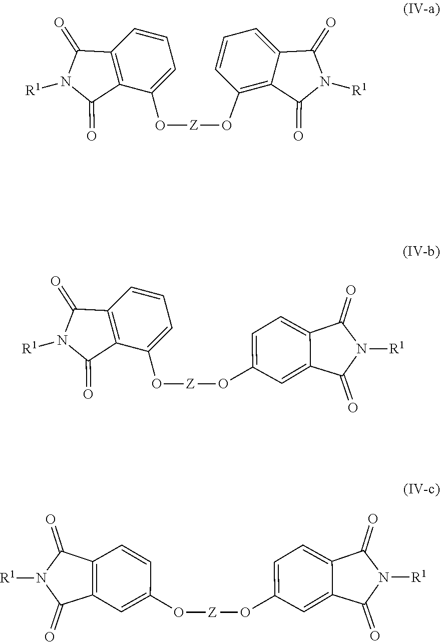

wherein R¹ is a C₁₋₁₃ alkyl group, or a C₁₋₄ alkyl group, preferably a methyl group, and Z is as described in formula (I). In some aspects, Z is a divalent group of formula (IIa), as described above, preferably 2,2-(4-phenylene)isopropylidene (i.e., the dihydroxy aromatic compound from which the dialkali metal salt is derived is 2,2-bis-(4-hydroxyphenyl)propane or bisphenol A). In some aspects, the aromatic bis(ether imide) comprises 3,3′-bisphenol-A-bis-N-methylphthalimide and optionally, 3,4′-bisphenol-A-bis-N-methylphthalimide, 4,4′-bisphenol-A-bis-N-methylphthalimide, or a combination thereof.

Methods for the manufacture of a polyetherimide are further disclosed. Advantageously, the disclosed methods can provide N—(C₁₋₁₃ alkyl)-3,3′-aromatic bis(ether imide) that it the major product rather than the minor product, as is the case with conventional methods. This allows control over the ratios of repeating units derived from N—(C₁₋₁₃ alkyl)-3,3′-aromatic bis(ether imide), repeating units derived from N—(C₁₋₁₃ alkyl)-3,4′-aromatic bis(ether imide), and repeating units derived from N—(C₁₋₁₃ alkyl)-4,4′-aromatic bis(ether imide). Therefore, by controlling the ratios of repeating units incorporated into the polyetherimides, the flow properties can be adjusted. In some aspects, it may be desirable for the polyetherimide to be essentially free (i.e., not detectable by analytical methods) of repeating units derived from N—(C₁₋₁₃ alkyl)-3,4′-aromatic bis(ether imide) and N—(C₁₋₁₃ alkyl)-4,4′-aromatic bis(ether imide). As an added advantage, polyetherimides derived from N—(C₁₋₁₃ alkyl)-3,3-aromatic bis(ether imide)s prepared according to the disclosed methods can have a lower halogen content as compared to polyetherimides prepared according to conventional methods. In certain aspects, the polyetherimides derived from N—(C₁₋₃ alkyl)-3,3-aromatic bis(ether imide)s prepared according to the disclosed methods are essentially halogen-free as defined by IEC 61249-2-21 or UL 74611.

In other aspects, it may be desirable for the polyetherimide to include a pre-determined amount of repeating units other than those derived from N—(C₁₋₁₃ alkyl)-3,3′-aromatic bis(ether imide) using the disclosed methods. As such, in addition to the N—(C₁₋₁₃ alkyl)-3,3′-aromatic bis(ether imide) prepared using the disclosed methods, a mixture of N—(C₁₋₁₃ alkyl)-3,3′-aromatic bis(ether imide), N—(C₁₋₁₃ alkyl)-3,4′-aromatic bis(ether imide), and N—(C₁₋₁₃ alkyl)-4,4′-aromatic bis(ether imide) prepared according to conventional methods can be added to achieve the pre-determined ratio of monomers.

The method for preparing the polyetherimides can include contacting the N—(C₁₋₁₃ alkyl)-3,3′-aromatic bis(ether imide) composition (IV) prepared according to the above-described method with a phthalic anhydride in the presence of a catalyst to provide an aromatic bis(ether phthalic anhydride) composition including 3,3′-aromatic bis(ether phthalic anhydride) of formula (V-a) and optionally, 3,4′aromatic bis(ether phthalic anhydride) of formula (V-b) and 4,4′-aromatic bis(ether phthalic anhydride) of formula (V-c)

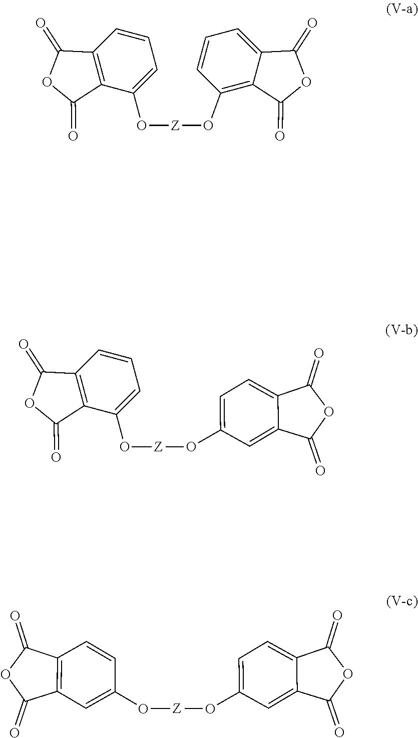

wherein Z is an aromatic C₆₋₂₄ monocyclic or polycyclic moiety optionally substituted with 1 to 6 C₁₋₈ alkyl groups, 1-8 halogen atoms, or a combination comprising thereof, as described above. In some aspects, Z is 2,2-(4-phenylene)isopropylidene. Illustrative examples of aromatic bis(ether phthalic anhydride)s include 3,3′-bis[4-(3,4-dicarboxyphenoxy)phenyl]propane dianhydride; 4,4-bis(3,4-dicarboxyphenoxy)diphenyl ether dianhydride; 4,4′-bis(3,4-dicarboxyphenoxy)diphenyl sulfide dianhydride; 4,4′-bis(3,4-dicarboxyphenoxy)benzophenone dianhydride; 4,4′-bis(3,4-dicarboxyphenoxy)diphenyl sulfone dianhydride; 2,2-bis[4-(2,3-dicarboxyphenoxy)phenyl]propane dianhydride; 4,4′-bis(2,3-dicarboxyphenoxy)diphenyl ether dianhydride; 4,4′-bis(2,3-dicarboxyphenoxy)diphenyl sulfide dianhydride; 4,4′-bis(2,3-dicarboxyphenoxy)benzophenone dianhydride; 4,4′-bis(2,3-dicarboxyphenoxy)diphenyl sulfone dianhydride; 4-(2,3-dicarboxyphenoxy)-4′-(3,4-dicarboxyphenoxy)diphenyl-2,2-propane dianhydride; 4-(2,3-dicarboxyphenoxy)-4′-(3,4-dicarboxyphenoxy)diphenyl ether dianhydride; 4-(2,3-dicarboxyphenoxy)-4′-(3,4-dicarboxyphenoxy)diphenyl sulfide dianhydride; 4-(2,3-dicarboxyphenoxy)-4′-(3,4-dicarboxyphenoxy)benzophenone dianhydride; and, 4-(2,3-dicarboxyphenoxy)-4′-(3,4-dicarboxyphenoxy)diphenyl sulfone dianhydride, as well as various combinations thereof.

The catalyst can be a tertiary amine. Among tertiary amines that can be used as catalysts are aliphatic tertiary amines such as triethylamine and tributylamine, cycloaliphatic tertiary amines such as N,N-diethyl-cyclohexylamine, and aromatic tertiary amines such as N,N-dimethylaniline. In some aspects, the catalyst comprises a C₁₋₂₀ trialkylamine, for example triethylamine, tributylamine, and the like, or a combination thereof. In an aspect, the catalyst is triethylamine. In some aspects, the contacting can occur in the presence of 0.5-15 mole percent of the catalyst with respect to the anhydride.

The 3,3′-aromatic bis(ether imide) composition and the phthalic anhydride are contacted under conditions that are generally known to be effective to provide the 3,3′-aromatic bis(ether phthalic anhydride) composition that includes 3,3′-aromatic bis(ether phthalic anhydride) and optionally, 3,4′-aromatic bis(ether phthalic anhydride) and 4,4′-aromatic bis(ether phthalic anhydride). For example, the phthalic anhydride can be present in a molar excess compared to the aromatic bis(ether imide), for example 3-8 molar excess of phthalic anhydride relative to aromatic bis(ether imide). The contacting can further be in the presence of a solvent, for example, an aromatic solvent including, but not limited to, benzene, toluene, xylene, chlorobenzene, and o-dichlorobenzene, preferably toluene. In some aspects, the solvent can comprise a solvent mixture, for example water and toluene. The contacting can be at a temperature of 100-300° C., or 100-280° C., or 100-250° C., or 110-240° C., or 120-230° C., or 130-220° C., or 150-210° C., or 150-250° C., or 170-260° C. The contacting can be at superatmospheric pressure, for example 200-700 pounds per square inch (psi), or 200-400 psi, or 200-600 psi, or 300-500 psi, or 300-600 psi, or 300-700 psi, or 400-600 psi, or 500-700 psi. The contacting of the aromatic bis(ether imide) and the phthalic anhydride can be carried out for 0.5-3 hours, preferably with agitation (e.g., stirring).

The method for manufacturing the polyetherimide further comprises contacting the 3,3′-aromatic bis(ether phthalic anhydride) composition of formula (V) with an organic diamine of formula (VI)

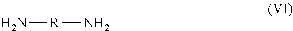

to provide the polyetherimide. In formula (VI), R is a substituted or unsubstituted divalent organic group, such as a substituted or unsubstituted C₆₋₂₀ aromatic hydrocarbon group, a substituted or unsubstituted straight or branched chain C₄₋₂₀ alkylene group, a substituted or unsubstituted C₃₋₈ cycloalkylene group, in particular a halogenated derivative of any of the foregoing. Specifically is in particular a divalent group of one or more of the following formulas

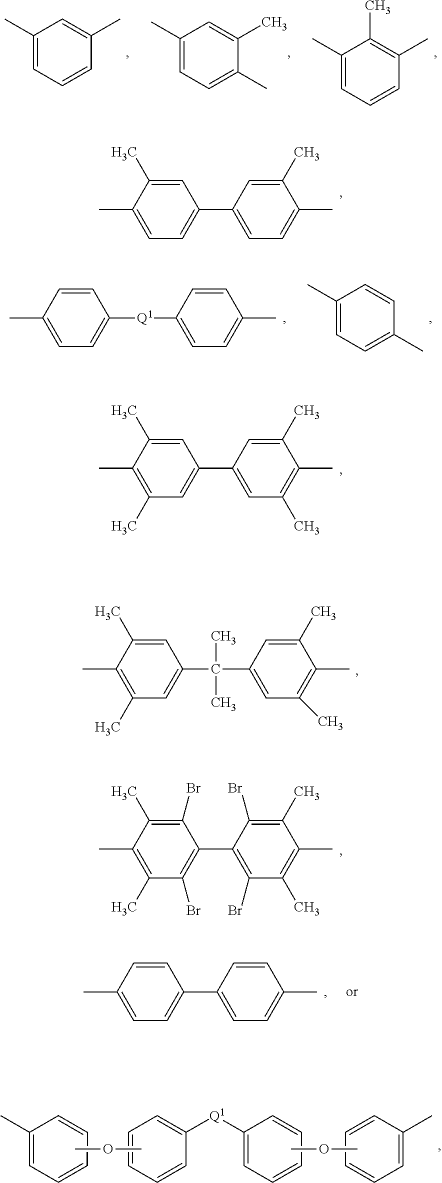

wherein Q¹ is —O—, —S—, —C(O)—, —SO₂—, —SO—, —P(R^{a})(═O)— wherein R^{a} is a C₁₋₈ alkyl or C₆₋₁₂ aryl, C_{y}H_{2y}— wherein y is an integer from 1-5 or a halogenated derivative thereof (which includes perfluoroalkylene groups), or —(C₆H₁₀)_{z}— wherein z is an integer from 1-4. In an aspect R is m-phenylene, p-phenylene, or a diarylene sulfone, in particular bis(4,4′-phenylene)sulfone.

Examples of organic diamines include 1,4-diaminobutane, 1,5-pentanediamine, 1,6-hexanediamine, 1,7-heptanediamine, 1,8-octanediamine, 1,9-nonanediamine, 1,10-decanediamine, 1,12-dodecanediamine, 1,18-octadecanediamine, 3-methylheptamethylenediamine, 4,4-dimethylheptanethylenediamine, 4-methylnonamethylenediamine, 5-methylnonamethylenediamine, 2,5-dimethylhexamethylenediamine, 2,5-dimethylheptamethylenediamine, 2, 2-dimethylpropylenediamine, N-methyl-bis(3-aminopropyl) amine, 3-methoxyhexamethylenediamine, 1,2-bis(3-aminopropoxy) ethane, bis(3-aminopropyl) sulfide, 1,4-cyclohexanediamine, bis-(4-aminocyclohexyl) methane, m-phenylenediamine, p-phenylenediamine, 2,4-diaminotoluene, 2,6-diaminotoluene, m-xylylenediamine, p-xylylenediamine, 2-methyl-4,6-diethyl-1,3-phenylene-diamine, 5-methyl-4,6-diethyl-1,3-phenylene-diamine, benzidine, 3,3′-dimethylbenzidine, 3,3′-dimethoxybenzidine, 1,5-diaminonaphthalene, bis(4-aminophenyl) methane, bis(2-chloro-4-amino-3,5-diethylphenyl) methane, bis(4-aminophenyl) propane, 2,4-bis(p-amino-t-butyl) toluene, bis(p-amino-t-butylphenyl) ether, bis(p-methyl-o-aminophenyl) benzene, bis(p-methyl-o-aminopentyl) benzene, 1, 3-diamino-4-isopropylbenzene, bis(4-aminophenyl) sulfide, bis-(4-aminophenyl) sulfone (also known as 4,4′-diaminodiphenyl sulfone (DDS)), and bis(4-aminophenyl) ether. C₁₋₄ alkylated or poly(C₁₋₄)alkylated derivatives of any of the foregoing can be used, for example a polymethylated 1,6-hexanediamine. Regioisomers of any of the foregoing can also be used. Combinations of these compounds can also be used. In some aspects the organic diamine is m-phenylenediamine, p-phenylenediamine, 4,4′-diaminodiphenyl sulfone, 3,4′-diaminodiphenyl sulfone, 3,3′-diaminodiphenyl sulfone, or a combination thereof.

The contacting of the 3,3′-aromatic bis(ether phthaiic anhydride) composition with the organic diamine can be in the presence of a solvent, for example, N-methylpyrrolidone, dimethylacetamide, dimethylformamide, cresol, veratrole, phenetole, dimethylsulfoxide, trichloromethane, acetone, methanol, ethanol, toluene, benzene, chlorobenzene, bromobenzene, dichlorobenzenes, trichlorobenzenes (e.g., 1,2,4-trichlorobenzene), xylene (including m-xylene, o-xylene, p-xylene, and combinations comprising at least one of the foregoing), anisole, ethylbenzene, propylbenzene, mesitylene, and the like, or a combination thereof. Sufficient solvent is generally utilized to provide a solids content of 1-90%, or 10-90%, or 30-90%, or 50-90%, or 70-90%, or 1-10%, or 10-30%, or 10-50%, or 10-70%, or 10-80%, or 20-40%, or 20-60%, or 20-80%, or 30-50%, or 30-70%, or 30-80%, or 40-60%, or 40-80%, or 50-80%. In some aspects, the solids content can be 15-60%.

In some aspects, the contacting can be in the presence of an endcapping agent. The endcapping agent limits molecular weight growth rate, and thus can be used to control molecular weight in the polyetherimide. Exemplary endcapping agents include certain monoamines (for example aniline), monoanhydrides (for example phthalic anhydride), and the like. In an aspect, the endcapping agent is phthalic anhydride, such that the resulting polyetherimide comprises phthalimide as an endcap to the polymer chain. It should be understood, however, that the polyetherimides disclosed herein can be produced having any desired weight average molecular weight (Mw) with any endcap.

The contacting of the 3,3′-aromatic bis(ether phthalic anhydride) composition with the organic diamine can be at a temperature of 100-250° C., or 120-230° C., or 150-210° C., or 150-250° C., and can be carried out for 0.5-10 hours, preferably with agitation (e.g. stirring). To avoid deleterious oxidation reactions, the contacting of the aromatic bis(ether phthalic anhydride) with the organic diamine can be blanketed under an inert gas. Examples of such gases are dry nitrogen, helium, argon and the like. Dry nitrogen can be preferred. The reaction can be run at atmospheric to super-atmospheric pressure.

Alternatively, a method for the manufacture of a polyetherimide comprises hydrolyzing the aromatic bis(ether imide) composition of formula (IV) prepared by the above method under conditions effective to provide the corresponding aromatic tetraacid composition including an aromatic tetraacid of formula (VII-a), and optionally, an aromatic tetraacid of formula (VII-b), an aromatic tetraacid of formula (VII-c), or a combination thereof,

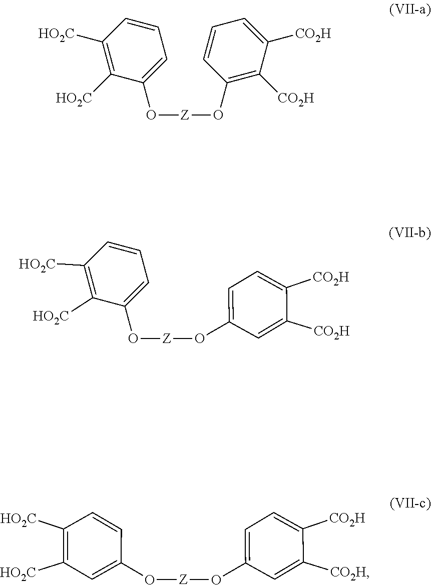

wherein Z is as defined above. In some aspects, Z is 2,2-(4-phenylene)isopropylidene.

Hydrolyzing the 3,3′-aromatic bis(ether imide) composition to provide the corresponding tetraacid can be under conditions effective to provide the aromatic tetraacid composition, for example, as described in U.S. Pat. No. 3,879,428. For example, the aromatic bis(ether imide) can be hydrolyzed in an aqueous alkaline solution, for example comprising an alkali metal hydroxide, preferably sodium hydroxide. Reaction time can vary from 1-24 hours or more depending upon reactants, degree of agitation, temperature, pressure, and the like. The organic amine by-product can be removed by standard procedures, such as steam distillation, decantation (when butyl-derived materials are used), and the like. In addition, the rate of hydrolysis is accelerated by carrying out the reaction at above atmospheric pressures, and at temperatures of 100-220° C. For example, hydrolysis can be at a temperature of 120-220° C., or 140-220° C., or 160-220° C., or 180-220° C., or 200-220° C., or 100-210° C., or 100-190° C., or 100-170° C., or 100-150° C., or 100-130° C. The hydrolysis can be at a pressure of 0 MPa-2 MPa. The hydrolyzed bis (ether imide) can be acidified with an acidic aqueous solution, for example comprising a mineral acid, for example sulfuric acid, hydrochloric acid, and the like, to provide the tetraacid.

The aromatic tetraacid composition can be condensed (i.e., dehydrated) under conditions effective to provide an aromatic bis(ether phthalic anhydride) composition of formula (V). Condensing the aromatic tetraacid composition to provide the corresponding aromatic aromatic bis(ether phthalic anhydride) composition can be under conditions effective to provide the aromatic bis(ether phthalic anhydride) composition. For example, the aromatic tetraacid can be condensed by refluxing in the presence of a dehydrating agent, for example acetic anhydride. In some aspects, a temperature of 100-225° C. and a pressure of 0 MPa-1 MPa can be used. The aromatic bis(ether phthalic anhydride) composition can optionally be isolated using any isolation techniques that are generally known, for example, filtration. Alternatively, the aromatic bis(ether phthalic anhydride) composition can be used directly for the preparation of the polyetherimide without further purification or isolation.

The method for the manufacture of the polyetherimide via the aromatic tetraacid composition (VII) can further comprise contacting the aromatic bis(ether phthalic anhydride) composition (V) (obtained by dehydrating the aromatic tetraacid composition as described above) with an organic diamine of formula (VI) to provide the polyetherimide. The contacting can be in the presence of a solvent or an endcapping agent as described above. Alternatively, in some aspects, the polyetherimide can be prepared by contacting the aromatic tetraacid composition (VII) directly with an organic diamine (VI) to provide the polyetherimide (i.e., dehydration of the tetraacid to provide the corresponding aromatic bis(ether phthalic anhydride) is not required).

The polyetherimide prepared according to either of the above-described methods for the manufacture of a polyetherimide can have a YI of less than 120, or less than 110, or less than 100, or less than 90, or less than 80, or less than 70, or less than 60, or less than 50. For example, the polyetherimide can have a YI of 40-120, or 40-110, or 40-100, or 40-90, or or 40-80, or 40-70, or 40-60, or 40-50. In some aspects, the polyetherimide can have a YI of 45-120, or 45-100, or 45-90, or 45-80, or 45-70, or 45-60, or 45-55. In other aspects, the polyetherimide can have a YI of 50-120, or 50-100, or 50-90, or 50-80, or 50-70, or 50-60 In still other aspects, the polyetherimide can have a YI of 60-120, or 60-100, or 60-90, or 60-80 or 60-70; or 70-120, or 70-100, or 70-90 or 70-80; or 80-120, or 80-100. In any of the foregoing aspect, the YI of the polyetherimide can be determined according to ASTM D1925, at a thickness of 3.2 millimeters.

The polyetherimide prepared according to either of the above-described methods for the manufacture of a polyetherimide can have a melt volume flow rate, measured at 337° C./6.6 kgf or 367° C./6.6 kgf, of 1 to 50 cubic centimeters per 10 minutes (cc/10 min), preferably 2 to 30 cc/10 min, as determined according to ASTM D1238.

The polyetherimides comprise repeating units of formula (VIII-a) and optionally, repeating units of formula (VIII-b), repeating units of formula (VIII-c), or a combination thereof,

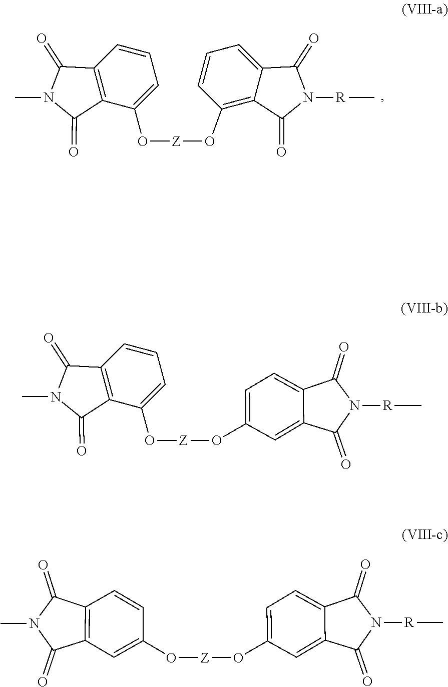

wherein Z is an aromatic C₆₋₂₄ monocyclic or polycyclic moiety optionally substituted with 1-6 C₁₋₈ alkyl groups, 1-8 halogen atoms, or a combination thereof, R is a C₆₋₂₀ aromatic hydrocarbon group or a halogenated derivative thereof, a straight or branched chain C₂₋₂₀ alkylene group or a halogenated derivative thereof, or a C₃₋₈ cycloalkylene group or halogenated derivative thereof, and wherein the polyetherimide comprises less than 20,000 ppm, less than 10,000 ppm, less than 5000 ppm, less than 2500 ppm, or less than 1000 ppm of repeating units of formula (VIII-b), repeating units of formula (VIII-c), or a combination thereof. The presence in ppm of repeating units of formula (VIII-b) and repeating units of formula (VIII-c) may be measured by NMR.

The polyetherimides prepared according to the above-described methods comprise repeating units of formula (VIII-a) and optionally, formula (VIII-b), formula (VIII-c), or a combination thereof, wherein Z is as defined in formula (I) and each R is the same or different, and is as defined in formula (VI). In an aspect, in formula (VIII) R is m-phenylene, p-phenylene, 4,4′-diaminodiphenyl sulfone, 3,4′-diaminodiphenyl sulfone, 3,3′-diaminodiphenyl sulfone, or a combination thereof and Z is a divalent group of formula (II-a) wherein Q is 2,2-isopropylidene.

An article comprising the polyetherimides prepared as described above is further disclosed. An article comprising the polyetherimides prepared as described above is further disclosed. Articles including the polyetherimide can be prepared by any number of methods including shaping, foaming, extruding, thermoforming, spinning, or molding. Articles can be in the form, for example, of fibers, hollow fibers, hollow tubes, sheets, films, multilayer sheets, multilayer films, molded parts, extruded profiles, coated parts, foams, filaments, or powders. In some embodiments, the article is a fiber, a film, a sheet, a foam, a filament, a molded article, an extruded article, or a powder.

Articles including the polyetherimide can be prepared by any number of methods including shaping, foaming, extruding, thermoforming, spinning, or molding. Articles can be in the form, for example, of fibers, hollow fibers, hollow tubes, sheets, films, multilayer sheets, multilayer films, molded parts, extruded profiles, coated parts, foams, filaments, or powders. In some aspects, the article is a fiber, a film, a sheet, a foam, a filament, a molded article, an extruded article, or a powder.

This disclosure is further illustrated by the following examples, which are non-limiting.

### EXAMPLES

### Example 1

Synthesis of 3-nitro-N-methyl phthalimide (3-NPI) from 3-nitro phthalic acid. The reaction was performed at a 5 g and 25 g scale, 3-nitro phthalic acid (3-NPAcid) was heated at 210° C. As the 3-NPAcid began to melt, the anhydride ring began to form and water was generated. After 1 h, the conversion of 3-NPAcid to 3-NP-Anhydride was complete and the temperature was reduced to 165° C. with stirring. Monomethylamine gas (MMA) was introduced into the reaction vessel. MMA reacts with the anhydride in a ring-opening reaction and the ring-opened intermediate undergoes ring-closure in situ, thus generating water. Complete conversion to 3-NPI was indicated when water was no longer produced by the reaction. The reaction mixture was cooled to provide a solid mass. The solid mass was flaked to provide flaked 3-NPI. The crude product was characterized by LC-MS and used in the next step without further purification. Suitable HPLC conditions may be found in U.S. Pat. No. 4,902,809.

TABLE 1

| | | | | % Purity |
|---|---|---|---|---|
| 3-NPAcid | MMA | 3-NPI Yield | M.P. | by LC-MS |
| 5 g | 2 (2.72 eq.) | 4 g (81.9%) | 107.3-109.8° C. | 93.62% |
| 25 g | 8 (2.17 eq.) | 20 g (81.9%) | 105.8-108.8° C. | 83.43% |

3-NPAcid (3-nitrophthalic acid),
MMA (monomethylamine),
3-NPI (3-nitrophthalimide)

Synthesis of 33′-bisphenol A bis(imide ether) (3,3-BPABI). The crude products obtained in the table above were carried forward in two batches. 3-NPI was dissolved in toluene at 75-85° C. Once the 3-NPI was completely dissolved, the solution was washed with a 2% sodium bicarbonate (aq) solution, followed by a water wash, and then the organic layer containing 3-NPI in toluene was dried with 1 mol % hexaethyl guanidinium chloride (HEG-Cl) until the moisture content was less than 200 ppm. The pre-dried 3-NPI (45.5 g, 0.217 mol) in toluene (275 mL) along with 1 mol % HEG-Cl and a solution of a pre-dried salt of BPA (BPANa2. 25 g, 0.1096 mol) in toluene (275 mL) were combined with stirring. The pre-dried salt of BPA was prepared as described in U.S. Pat. Nos. 7,902,407 and 395,786. The reaction proceeded to completion in 1 h. An aliquot was removed for LC-MS analysis. The results for the first batch are labeled as 1-RM and the results for the second batch are labeled a 1-2-RM. The reaction mixture was washed with water, followed by 2% NaHCO₃ (aq.) to remove unreacted bisphenol, 3-NPI, and other impurities. Toluene was removed and the isolated solid was characterized. The LC-MS results for the first batch are labeled as 1-P and the results for the second batch are labeled as 2-P.

In Table 2, the abbreviations are as follows: 3-NPI (3-nitrophthalimide), BPA (bisphenol A), MI (mono imide), 3,3′-BI (3,3′-bis (ether imide)), 3,4-BI (3,4-bis (ether imide)), and 4,4′-BI (4,4′-bis (ether imide). The amounts of each component were measured using LC-MS.

TABLE 2

| | 3-NPI | BPA | MI | 3,3-BI | 3,4-BI | 4,4-BI | | |
|---|---|---|---|---|---|---|---|---|
| Sample | (ppm) | (ppm) | (ppm) | (wt %) | (ppm) | (ppm) | Yield | YI |
| 1-RM | 510 | 6000 | 23,200 | 94.09 wt % | 530 | 100 | 50 g | 9.47 |
| 1-P | ND | ND | 87.1 wt % | 99.96 wt % | ND | 300 | (83.7%) | |
| 2-RM | 10,300 | 26,000 | 2800 | 98.38 wt % | 2000 | ND | 45 g | 5 |
| 2-P | 13 | 27 | 501 | 99.75 | 1800 | 100 | (75%) | |

### Comparative Example 1

Five batches of aromatic bis (ether imide) (“BI”) were prepared using 3-chlorophthalic imide as starting material. The abbreviations used in Table 3 are defined as follows: 3-ClPI (3-chlorophthalimide), BPA (bisphenol A), MI (mono imide), 3,3′-BI (3,3′-bis (ether imide)), 3,4-BI (3,4-bis (ether imide)), and 4,4′-BI (4,4′-bis (ether imide)). The amounts of each component were measured using LC-MS.

TABLE 3

| | 3-ClPI | BPA | MI | 3,3-BI | 3,4-BI | 4,4-BI | |
|---|---|---|---|---|---|---|---|
| Batches | (ppm) | (wt %) | (ppm) | (wt %) | (ppm) | (ppm) | YI |
| C1 | 24.3 | ND | 189.2 | 96.66 | 30,140 | 1800 | 14.93 |
| C2 | 21.7 | ND | 248.2 | 96.94 | 29,600 | 800 | 14.25 |
| C3 | 13.5 | ND | 143.2 | 96.94 | 29,700 | 800 | 13.51 |
| C4 | ND | ND | 169.2 | 97.13 | 26,100 | 2500 | 14.85 |
| C5 | ND | ND | 214.3 | 96.95 | 29,700 | 600 | 14.17 |

Conversion of 3-chlorophthalic anhydride to 3-chlorophthalimide (3-ClPI) can be accomplished using two methods.

3-Chlorophthalic anhydride was dissolved in ortho-dichlorobenzene. To this solution was added an aqueous monomethyl amine solution with stirring. The reaction mixture was stirred with heating less than 70° C. while removing the bulk water. Once the bulk water was removed (145° C.), drying was continued to remove water generated due to ring closure and solvent was removed by distillation with heating (180° C.) to provide 3ClPI.

Alternatively, 3-chlorophthalic anhydride was melted and MMA gas was passed through the melt to provide the ring-opened amide intermediate. In situ ring-closure provided the 3ClPI.

Preparation of 3,3-BPABI from 3ClPI. 3ClPI (410 kg) was dissolved in toluene (3800 L). The solution was dried with 4-6 mol % HEG-Cl to reduce the moisture content to less than 200 ppm. A BPA salt solution (1400 kg) in toluene was added portion-wise to the dried solution. The reaction proceeded for 6-10 hours to ensure maximum conversion. The reaction mixture was washed with a 2% NaHCO₃ (aq.) to remove unreacted starting material and impurities.

In reaction batches C1-C5, although the 3-ClPI was almost completely consumed or completely consumed, the reactions resulted in significantly higher levels of 3,4-BI and 4,4-BI than the process of Example 1 due to the presence of 4-chlorophthalic anhydride in the starting material.

The procedure described above was repeated on commercial scale, starting with 400 kg 3-NPI (1.89 kmol) and 55 kg MMA (1.77 kmol). After the reaction was complete, the reaction mixture was washed twice with 2% NaHCO₃ (aq.). The resulting product 3-NPI (367 kg) was characterized by LC-MS and carried on to the next step without further purification.

Analysis of the product is summarized in Table 4. “ND” means not detected. The abbreviations used in Table 4 are defined as follows: 3-NPA (3-nitrophthalic acid), 3-NPI (3-nitrophthalimide), PI (phthalimide), DNPI (4-hydroxy-3,5-dinitro-N-methylphthalimide), MI (mono imide), 3,3′-BI (3,3′-bis (ether imide)), 3,4′-BI (3,4′-bis (ether imide)), and 4,4′-BI (4,4′-bis (ether imide)). The amounts of each component were measured using LC-MS.

TABLE 4

| | | | | | Max | moisture |
|---|---|---|---|---|---|---|
| 3NPA | DNPI | PI | 3-NPI | 4NPI | Unimp. | content |
| <2.0% | <0.3% | <0.5% | >95% | <1% | <2% | <0.5% |
| 0.01 | ND | ND | 98.0 | ND | 0.84 | 0.12 |

As shown in the table above, 3-NPI is almost completely consumed and side-products 4-hydroxy-3,5-dinitro-N-methylphthalimide, 4-nitrophthalimide, and phthalimide were not detected.

The 3-NPI (367 kg, 1.78 kmol) was dissolved in toluene (3.1 kL). The temperature of the solution was increased to 75-85° C. to completely dissolve the 3-NPI. The heated solution was washed with 20 kg of 2% NaHCO₃, followed by 1.5 L water. HEG-Cl (2 mol %) was added to the resulting organic layer to dry the material. BPANa2 in toluene (total volume 3.1 L) was added in two portions. After completion of the reaction, the reaction mixture was washed with water, followed by 1% NaOH (aq.). The solvent was removed and the product was characterized by LC-MS. The N-methyl-3,3-aromatic bis(ether imide) was obtained in a 99.98% yield. The N-methyl-3,4′-aromatic bis(ether imide) and N-methyl-4,4′-aromatic bis(ether imide) were present at 700 ppm and 500 ppm, respectively.

This disclosure further encompasses the following aspects.

Aspect 1a. A method for the preparation of a 3-nitro-N—(C₁₋₁₃ alkyl)phthalimide composition) comprising reacting a 3-nitro phthalic acid optionally in the presence of a solvent, under conditions effective to provide 3-nitro-phthalic anhydride and water, and wherein the water is removed from the reaction mixture during the reacting, and contacting 3-nitro-phthalic anhydride with a C₁₋₁₃ alkylamine optionally in the presence of a solvent under conditions effective to provide the 3-nitro-N—(C₁₋₁₃ alkyl)phthalimide composition comprising 3-nitro-N—(C₁₋₁₃ alkyl)phthalimide and optionally, 4-nitro-N—(C₁₋₁₃ alkyl)phthalimide.

Aspect 1 b. The method of aspect 1, wherein the 3-nitro-N—(C₁₋₁₃ alkyl)phthalimide composition comprises less than 20,000 ppm, less than 10,000 ppm, less than 5000 ppm, less than 2500 ppm, or less than 1000 ppm of the 4-nitro-N—(C₁₋₁₃ alkyl)phthalimide, as determined by LC-MS.

Aspect 2. The method of aspect 1a or 1 b, wherein the percent yield of the 3-nitro-N—(C₁₋₁₃ alkyl)phthalimide composition is at least 60%, 65%, 70%, 75%, or 80%, based on the weight of 3-nitro phthalic acid.

Aspect 2a. The method of aspect 1a or 1b, wherein the percent yield of the 3-nitro-N—(C₁₋₁₃ alkyl)phthalimide composition is at least 60%, 65%, or 70%, based on 25 g or less of 3-nitro phthalic acid, or based on 25 g of 3-nitro phthalic acid.

Aspect 2b. The method of aspect 1a or 1b, wherein the percent yield of the 3-nitro-N—(C₁₋₁₃ alkyl)phthalimide composition is at least 70%, 75%, or 80%, based on 5 g or less of 3-nitro phthalic acid, or based on 5 g of 3-nitro phthalic acid.

Aspect 3a. A method for the preparation of an N—(C₁₋₁₃ alkyl)-3,3′-aromatic bis(ether imide) composition comprising reacting a dialkali metal salt of a dihydroxy aromatic compound with the 3-nitro-N—(C₁₋₁₃ alkyl)phthalimide composition prepared according to aspect 1a or 1b under conditions effective to form a product mixture comprising the N—(C₁₋₁₃ alkyl)-3,3′-aromatic bis(ether imide) composition comprising N—(C₁₋₁₃ alkyl)-3,3′-aromatic bis(ether imide) and optionally, N—(C₁₋₁₃ alkyl)-3,4′-aromatic bis(ether imide), N—(C₁₋₃ alkyl)-4,4′-aromatic bis(ether imide), or a combination thereof.

Aspect 3b. The method of aspect 3a, wherein the N—(C₁₋₁₃ alkyl)-3,4′-aromatic bis(ether imide), the N—(C₁₋₁₃ alkyl)-4,4′-aromatic bis(ether imide), or a combination thereof comprises less than 20,000 ppm, less than 10,000 ppm, less than 5000 ppm, less than 2500 ppm, or less than 1000 ppm of the N—(C₁₋₁₃ alkyl)-3,3′-aromatic bis(ether imide) composition, as determined by LC-MS.

Aspect 4. The method of any of the preceding aspects, wherein the reacting of the 3-nitro phthalic acid to provide the 3-nitro-phthalic anhydride is performed with heating in the absence of a solvent, the reacting of the 3-nitro-phthalic anhydride with the C₁₋₁₃ alkylamine to provide the 3-nitro-N—(C₁₋₁₃ alkyl)phthalimide composition is performed in the absence of solvent, or a combination thereof.

Aspect 5. The method of any one of aspects 1-4, wherein the reacting of the 3-nitro phthalic acid to provide the 3-nitro-phthalic anhydride and the reacting of the 3-nitro-phthalic anhydride with the C₁₋₁₃ alkylamine to provide the 3-nitro-N—(C₁₋₁₃ alkyl)phthalimide composition is a continuous process.

Aspect 6. The method of any one of aspects 3a, 3b, 4, or 5, wherein the dialkali metal salt of a dihydroxy aromatic compound is of the formula

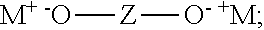

the 3-nitro-N—(C₁₋₁₃ alkyl)phthalimide is of the formula (III-a) and the 4-nitro-N—(C₁₋₁₃ alkyl)phthalimide is of the formula (III-b)

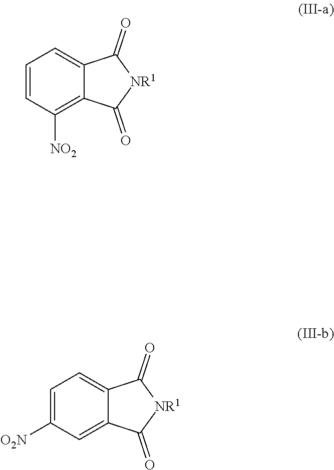

the N—(C₁₋₁₃ alkyl)-3,3′-aromatic bis(ether imide) is of the formula (IV-a), the N—(C₁₋₁₃ alkyl)-3,4′-aromatic bis(ether imide) is of the formula (IV-b), and the N—(C₁₋₁₃ alkyl)-4,4′-aromatic bis(ether imide) is of the formula (IV-c)

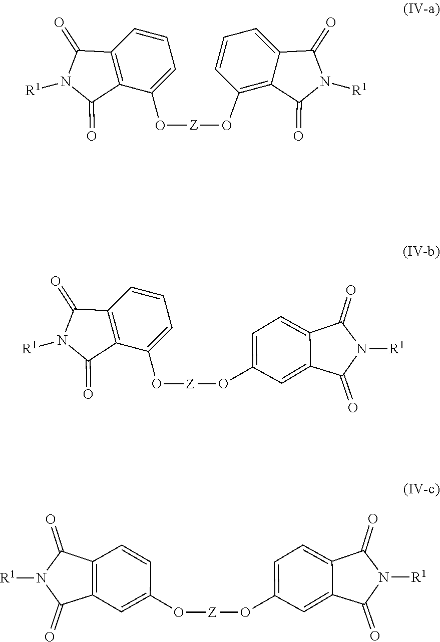

wherein in the foregoing formulas, M is an alkali metal; Z is an aromatic C₆₋₂₄ monocyclic or polycyclic moiety optionally substituted with 1-6 C₁₋₈ alkyl groups, 1-8 halogen atoms, or a combination thereof; and R¹ is a monovalent C₁₋₁₃ alkyl group, preferably methyl.

Aspect 7. The method of aspect 5 or 6, wherein Z is a divalent group of the formula

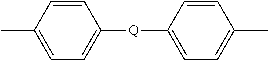

wherein Q is —O—, —S—, —C(O)—, —SO₂—, —SO—, or —C_{y}H_{2y}— wherein y is an integer from 1-5 or a halogenated derivative thereof, preferably wherein Z is 2,2-(4-phenylene)isopropylidene.

Aspect 8. The method of any one of aspects 3a, 3b, 4, 5, 6 or 7, further comprising isolating the 3,3-aromatic bis(ether imide) composition, wherein the isolated aromatic bis(ether imide) has a yellowness index of less than 10 as determined according to ASTM D-1925 at a thickness of 3.2 mm.

Aspect 9a. The 3-nitro-N—(C₁₋₁₃ alkyl)phthalimide) composition prepared according to the method of Aspect 1a or 1b.

Aspect 9b. A 3-nitro-N—(C₁₋₁₃ alkyl)phthalimide composition comprising 3-nitro-N—(C₁₋₁₃ alkyl)phthalimide and optionally, 4-nitro-N—(C₁₋₁₃ alkyl)phthalimide, wherein the 3-nitro-N—(C₁₋₁₃ alkyl)phthalimide composition comprises less than 20,000 ppm, less than 10,000 ppm, less than 5000 ppm, less than 2500 ppm, or less than 1000 ppm of 4-nitro-N—(C₁₋₁₃ alkyl)phthalimide.

Aspect 10a. A 3,3′-aromatic bis(ether imide) composition comprising 3,3′-aromatic bis(ether imide) and optionally, 3,4′-aromatic bis(ether imide), 4,4′-aromatic bis(ether imide), or a combination thereof, wherein the 3,3′-aromatic bis(ether imide) composition comprises less than 20,000 ppm, less than 10,000 ppm, less than 5000 ppm, less than 2500 ppm, or less than 1000 ppm of 3,4′-aromatic bis(ether imide), or 4,4′-aromatic bis(ether imide.

Aspect 10b. The 3,3′-aromatic bis(ether imide) composition prepared according to the method of any one of aspects 3-8.

Aspect 11. A method for the manufacture of a polyetherimide, the method comprising contacting the 3,3′-aromatic bis(ether imide) composition prepared by the method of any one of aspects 3-8 with a phthalic anhydride in the presence of a catalyst and under conditions effective to provide a 3,3′-bis(ether phthalic anhydride) composition comprising 3,3′-aromatic bis(ether phthalic anhydride) of formula (V-a) and optionally, 3,4′-aromatic bis(ether phthalic anhydride) of formula (V-b), 4,4′-aromatic bis(ether phthalic anhydride) of formula (V-c), or a combination thereof

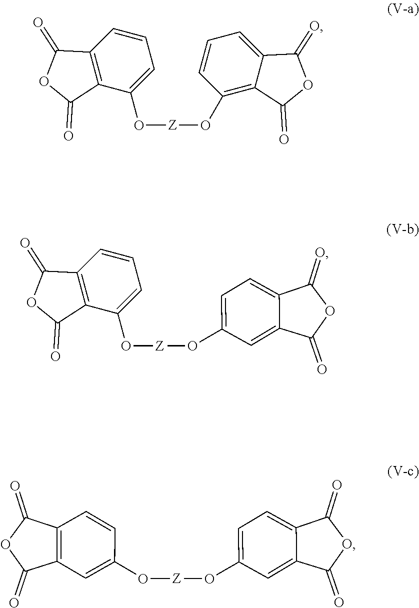

wherein Z is an aromatic C₆₋₂₄ monocyclic or polycyclic moiety optionally substituted with 1-6 C₁₋₈ alkyl groups, 1-8 halogen atoms, or a combination thereof; contacting the N—(C₁₋₁₃ alkyl)-3,3′-bis(ether phthalic anhydride) composition with an organic diamine of the formula H₂N—R—NH₂
wherein R is a C₆₋₂₀ aromatic hydrocarbon group or a halogenated derivative thereof, a straight or branched chain C₂₋₂₀ alkylene group or a halogenated derivative thereof, a C₃₋₈ cycloalkylene group or halogenated derivative thereof.

Aspect 12. A method for the manufacture of a polyetherimide, the method comprising, hydrolyzing aromatic bis(ether imide) prepared by the method of any one of aspects 3-8 under conditions effective to provide the corresponding a aromatic bis(ether tetraacid) composition comprising an aromatic bis(ether tetracid) of formula (VII-a) and optionally, an aromatic bis(ether tetracid) of formula (VII-b), an aromatic bis(ether tetracid) of formula (VII-c), or a combination thereof,

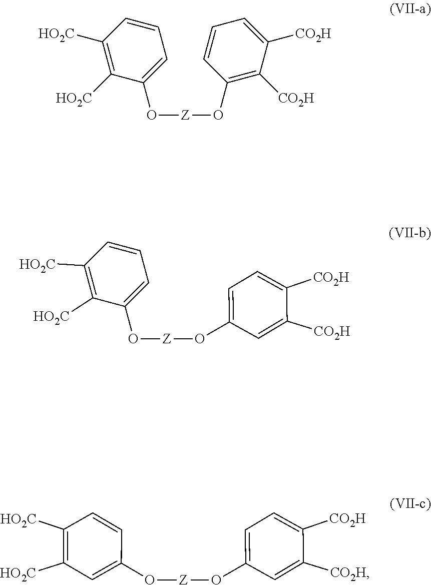

condensing the aromatic bis(ether tetraacid) composition under conditions effective to provide a 3,3′-aromatic bis(ether phthalic anhydride) composition comprising 3,3′-aromatic bis(ether phthalic anhydride) and optionally, a 3,4′-aromatic bis(ether phthalic anhydride), 4,4′-aromatic bis(ether phthalic anhydride), or a combination thereof,

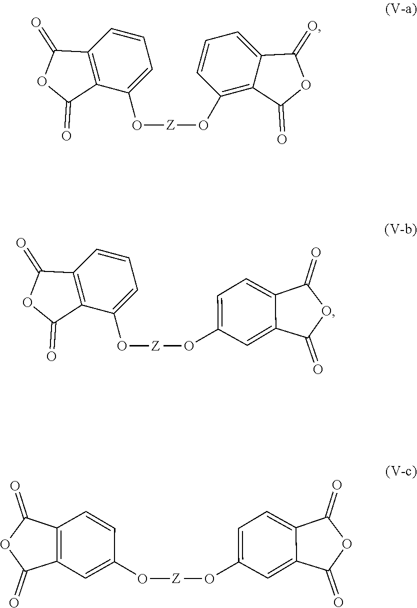

wherein Z is an aromatic C₆₋₂₄ monocyclic or polycyclic moiety optionally substituted with 1-6 C₁₋₈ alkyl groups, or 1-8 halogen atoms, or a combination thereof; and contacting the 3,3′-bis(ether phthaiic anhydride) composition with an organic diamine of the formula (H₂N—R—NH₂) wherein R is a C₆₋₂₀ (aromatic hydrocarbon group or a halogenated derivative thereof, a straight or branched chain C₂₋₂₀ alkylene group or a halogenated derivative thereof, or a C₃₋₈ cycloalkylene group or halogenated derivative thereof.

Aspect 13a. A polyetherimide comprising repeating units of formula (VIII-a) and optionally, repeating units of formula (VIII-b), repeating units of formula (VIII-c), or a combination thereof,

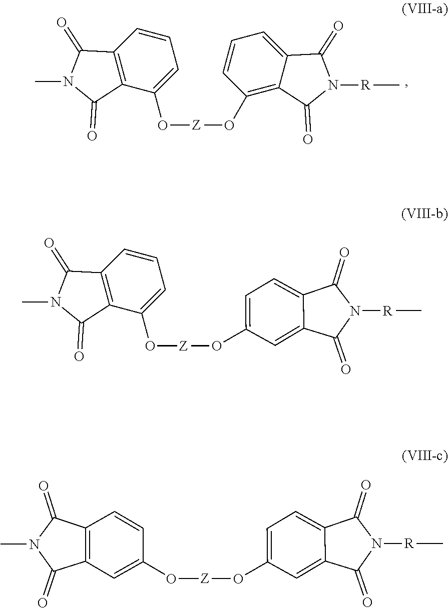

wherein Z is an aromatic C₆₋₂₄ monocyclic or polycyclic moiety optionally substituted with 1-6 C₁₋₈ alkyl groups, 1-8 halogen atoms, or a combination thereof, R is a C₆₋₂₀ aromatic hydrocarbon group or a halogenated derivative thereof, a straight or branched chain C₂₋₂₀ alkylene group or a halogenated derivative thereof, or a C₃₋₈ cycloalkylene group or halogenated derivative thereof, and wherein the polyetherimide comprises less than 20,000 ppm, less than 10,000 ppm, less than 5000 ppm, less than 2500 ppm, or less than 1000 ppm of repeating units of formula (VIII-b), repeating units of formula (VII-c), or a combination thereof.

Aspect 13b. The polyetherimide prepared according to the method of aspect 11 or 12.

Aspect 14. An article comprising the polyetherimide of aspect 13a or 13b, wherein the article is in the form of a fiber, a film, a sheet, a foam, a filament, a molded article, an extruded article, or a powder.

Aspect 15. A method of manufacturing the article of aspect 14, comprising molding, casting, or extruding the composition to provide the article.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt %, or, more specifically, 5 wt % to 20 wt %”, is inclusive of the endpoints and all intermediate values of the ranges of “5 wt % to 25 wt %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some aspects”, “an aspect”, and so forth, means that a particular element described in connection with the aspect is included in at least one aspect described herein, and may or may not be present in other aspects. In addition, it is to be understood that the described elements may be combined in any suitable manner in the various aspects. A “combination thereof” is open and includes any combination comprising at least one of the listed components or properties optionally together with a like or equivalent component or property not listed

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“—”) that is not between two letters or symbols is used to indicate a point of attachment for a substituent. For example, —CHO is attached through carbon of the carbonyl group.

The term “alkyl” means a branched or straight chain, unsaturated aliphatic hydrocarbon group, e.g., methyl, ethyl, n-propyl, i-propyl, n-butyl, s-butyl, t-butyl, n-pentyl, s-pentyl, and n- and s-hexyl. “Akenyl” means a straight or branched chain, monovalent hydrocarbon group having at least one carbon-carbon double bond (e.g., ethenyl (—HC═CH₂)). “Alkoxy” means an alkyl group that is linked via an oxygen (i.e., alkyl-O—), for example methoxy, ethoxy, and sec-butyloxy groups. “Alkylene” means a straight or branched chain, saturated, divalent aliphatic hydrocarbon group (e.g., methylene (—CH₂—) or, propylene (—(CH₂)₃—)) “Cycloalkylene” means a divalent cyclic alkylene group, —CₙH₂ₙ₋ₓ, wherein x is the number of hydrogens replaced by cyclization(s). “Cycloalkenyl” means a monovalent group having one or more rings and one or more carbon-carbon double bonds in the ring, wherein all ring members are carbon (e.g., cyclopentyl and cyclohexyl). “Aryl” means an aromatic hydrocarbon group containing the specified number of carbon atoms, such as phenyl, tropone, indanyl, or naphthyl. “Arylene” means a divalent aryl group. “Alkylarylene” means an arylene group substituted with an alkyl group. “Arylalkylene” means an alkylene group substituted with an aryl group (e.g., benzyl). The prefix “halo” means a group or compound including one more of a fluoro, chloro, bromo, or iodo substituent. A combination of different halo groups (e.g., bromo and fluoro), or only chloro groups can be present. The prefix “hetero” means that the compound or group includes at least one ring member that is a heteroatom (e.g., 1, 2, or 3 heteroatom(s)), wherein the heteroatom(s) is each independently N, O, S, Si, or P. “Substituted” means that the compound or group is substituted with at least one (e.g., 1, 2, 3, or 4) substituents that can each independently be a C₁₋₉ alkoxy, a C₁₋₉ haloalkoxy, a nitro (—NO₂), a cyano (—CN), a C₁₋₆ alkyl sulfonyl (—S(═O)₂-alkyl), a C₆₋₁₂ aryl sulfonyl (—S(═O)₂-aryl) a thiol (—SH), a thiocyano (—SCN), a tosyl (CH₃C₆H₄SO₂—), a C₃₋₁₂ cycloalkyl, a C₂₋₁₂ alkenyl, a C₅₋₁₂ cycloalkenyl, a C₆₋₁₂ aryl, a C₇₋₁₃ arylalkylene, a C₄₋₁₂ heterocycloalkyl, and a C₃₋₁₂ heteroaryl instead of hydrogen, provided that the substituted atom's normal valence is not exceeded. The number of carbon atoms indicated in a group is exclusive of any substituents. For example —CH₂CH₂CN is a C₂ alkyl group substituted with a nitrile.

While particular aspects have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for the preparation of a 3-nitro-N—(C₁₋₁₃ alkyl)phthalimide composition) comprising:
reacting 3-nitro phthalic acid optionally in the presence of a solvent, under conditions effective to provide a reaction mixture comprising 3-nitro-phthalic anhydride and water, and wherein the water is removed from the reaction mixture during the reacting;
contacting the 3-nitro-phthalic anhydride with a C₁₋₁₃ alkylamine optionally in the presence of a solvent to provide the 3-nitro-N—(C₁₋₁₃ alkyl)phthalimide composition comprising 3-nitro-N—(C₁₋₁₃ alkyl)phthalimide and optionally, 4-nitro-N—(C₁₋₁₃ alkyl)phthalimide.

2. The method of claim 1, wherein the percent yield of the 3-nitro-N—(C₁₋₁₃ alkyl)phthalimide composition is at least 60%, based on the weight of 3-nitro phthalic acid.

3. A method for the preparation of an N—(C₁₋₁₃ alkyl)-3,3′-aromatic bis(ether imide) composition comprising:
preparing the 3-nitro-N—(C₁₋₁₃ alkyl)phthalimide composition) according to claim 1; and
contacting a dialkali metal salt of a dihydroxy aromatic compound with the 3-nitro-N—(C₁₋₁₃ alkyl)phthalimide composition under conditions effective to form a product mixture comprising the N—(C₁₋₁₃ alkyl)-3,3′-aromatic bis(ether imide) composition comprising N—(C₁₋₁₃ alkyl)-3,3′-aromatic bis(ether imide) and optionally, N—(C₁₋₁₃ alkyl)-3,4′-aromatic bis(ether imide), N—(C₁₋₁₃ alkyl)-4,4′-aromatic bis(ether imide), or a combination thereof.

4. The method of claim 3, wherein
the reacting of the 3-nitro phthalic acid to provide the 3-nitro-phthalic anhydride is performed with heating in the absence of a solvent, or
the contacting of the 3-nitro-phthalic anhydride with the C₁₋₁₃ alkylamine to provide the 3-nitro-N—(C₁₋₁₃ alkyl)phthalimide composition is performed in the absence of solvent,
or a combination thereof.

5. The method of claim 1, wherein the reacting of the 3-nitro phthalic acid to provide the 3-nitro-phthalic anhydride and the contacting of the 3-nitro-phthalic anhydride with the C₁₋₁₃ alkylamine to provide the 3-nitro-N—(C₁₋₁₃ alkyl)phthalimide composition is a continuous process.

6. The method of claim 3, wherein
the dialkali metal salt of a dihydroxy aromatic compound is of the formula 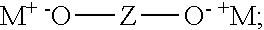
the 3-nitro-N—(C₁₋₁₃ alkyl)phthalimide is of the formula (III-a) and the 4-nitro-N—(C₁₋₁₃ alkyl)phthalimide is of the formula (III-b) 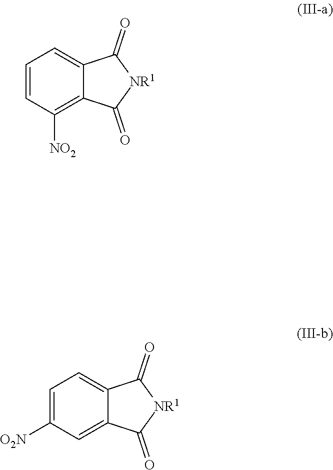
the N—(C₁₋₁₃ alkyl)-3,3′-aromatic bis(ether imide) is of the formula (IV-a), the N—(C₁₋₁₃ alkyl)-3,4′-aromatic bis(ether imide) is of the formula (IV-b), and the N—(C₁₋₁₃ alkyl)-4,4′-aromatic bis(ether imide) is of the formula (IV-c) 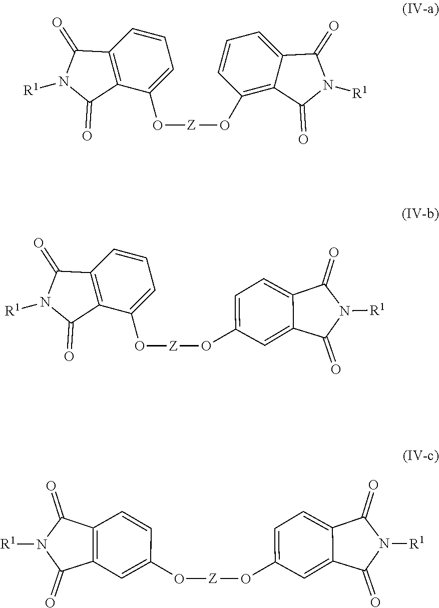
wherein in the foregoing formulas,
M is an alkali metal;
Z is an aromatic C₆₋₂₄ monocyclic or polycyclic moiety optionally substituted with 1-6 C₁₋₈ alkyl groups, 1-8 halogen atoms, or a combination thereof; and
R¹ is a monovalent C₁₋₁₃ alkyl group.

7. The method of claim 5, wherein Z is a divalent group of the formula 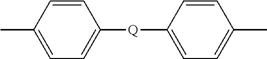
wherein Q is —O—, —S—, —C(O)—, —SO₂—, —SO—, or —C_{y}H_{2y}—, or a halogenated derivative thereof, and wherein y is an integer from 1-5.

8. The method of claim 3, further comprising isolating the 3,3-aromatic bis(ether imide) composition, wherein the isolated aromatic bis(ether imide) has a yellowness index of less than 10 as determined according to ASTM D-1925 at a thickness of 3.2 mm.

9. A 3-nitro-N—(C₁₋₁₃ alkyl)phthalimide composition comprising 3-nitro-N—(C₁₋₁₃ alkyl)phthalimide and optionally, 4-nitro-N—(C₁₋₁₃ alkyl)phthalimide, wherein the 3-nitro-N—(C₁₋₁₃ alkyl)phthalimide composition comprises less than 20,000 ppm of 4-nitro-N—(C₁₋₁₃ alkyl)phthalimide.

10. A 3,3′-aromatic bis(ether imide) composition comprising 3,3′-aromatic bis(ether imide) and optionally, 3,4′-aromatic bis(ether imide), 4,4′-aromatic bis(ether imide), or a combination thereof, wherein the 3,3′-aromatic bis(ether imide) composition comprises less than 20,000 ppm, of 3,4′-aromatic bis(ether imide), 4,4′-aromatic bis(ether imide), or a combination thereof.

11. A method for the manufacture of a polyetherimide, the method comprising:
preparing the N—(C₁₋₁₃ alkyl)-3,3′-aromatic bis(ether imide) composition according to claim 3; and
contacting the 3,3′-aromatic bis(ether imide) composition with a phthalic anhydride in the presence of a catalyst and under conditions effective to provide a 3,3′-bis(ether phthalic anhydride) composition comprising 3,3′-aromatic bis(ether phthalic anhydride) of formula (V-a) and optionally, 3,4′-aromatic bis(ether phthalic anhydride) of formula (V-b), 4,4′-aromatic bis(ether phthalic anhydride) of formula (V-c), or a combination thereof, 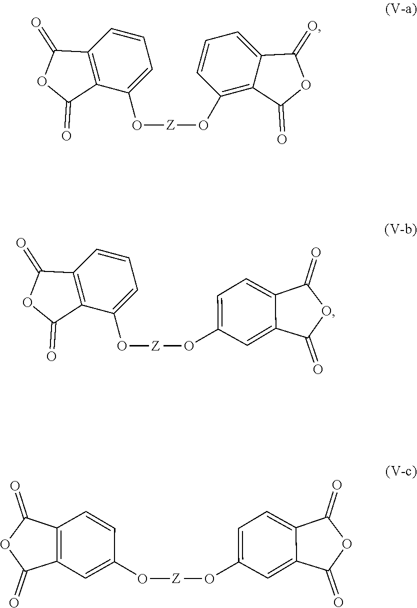
wherein Z is an aromatic C₆₋₂₄ monocyclic or polycyclic moiety optionally substituted with 1-6 C₁₋₈ alkyl groups, 1-8 halogen atoms, or a combination thereof;
contacting the 3,3′-bis(ether phthalic anhydride) composition with an organic diamine of the formula 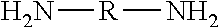
wherein R is a C₆₋₂₀ aromatic hydrocarbon group or a halogenated derivative thereof, a straight or branched chain C₂₋₂₀ alkylene group or a halogenated derivative thereof, or a C₃₋₈ cycloalkylene group or halogenated derivative thereof.

12. A method for the manufacture of a polyetherimide, the method comprising:
preparing the N—(C₁₋₁₃ alkyl)-3,3′-aromatic bis(ether imide) composition according to claim 3; and
hydrolyzing aromatic bis(ether imide) composition under conditions effective to provide the corresponding an aromatic bis(ether tetraacid) composition comprising an aromatic bis(ether tetracid) of formula (VII-a) and optionally, an aromatic bis(ether tetracid) of formula (VII-b), an aromatic bis(ether tetracid) of formula (VII-c), or a combination thereof, 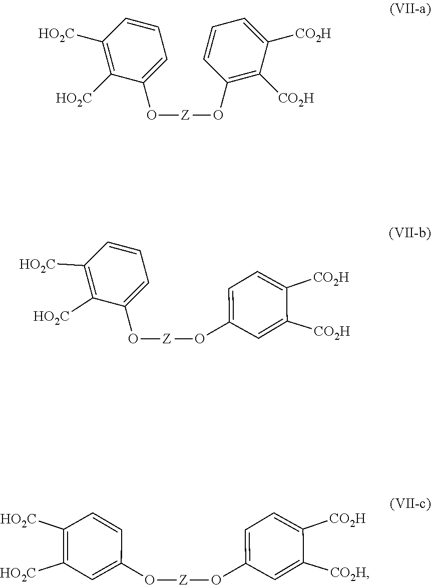
condensing the aromatic bis(ether tetraacid) composition under conditions effective to provide an aromatic bis(ether phthalic anhydride) composition comprising 3,3′-aromatic bis(ether phthalic anhydride) of formula (V-a) and optionally, 3,4′-aromatic bis(ether phthalic anhydride) of formula (V-b), 4,4′-aromatic bis(ether phthalic anhydride) of formula (V-c), or a combination thereof, 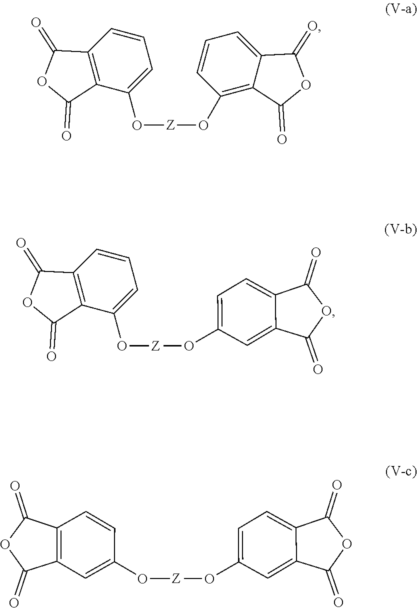
wherein Z is an aromatic C₆₋₂₄ monocyclic or polycyclic moiety optionally substituted with 1-6 C₁₋ₛ alkyl groups, or 1-8 halogen atoms; and
contacting the 3,3′-bis(ether phthalic anhydride) composition with an organic diamine of the formula 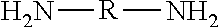
wherein R is a C₆₋₂₀ aromatic hydrocarbon group or a halogenated derivative thereof, a straight or branched chain C₂₋₂₀ alkylene group or a halogenated derivative thereof, or a C₃₋₈ cycloalkylene group or halogenated derivative thereof.

13. A polyetherimide comprising repeating units of formula (VIII-a) and optionally, repeating units of formula (VIII-b), repeating units of formula (VIII-c), or a combination thereof, 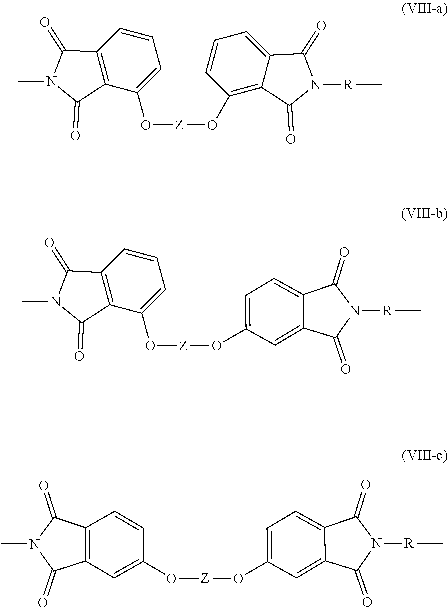
wherein
Z is an aromatic C₆₋₂₄ monocyclic or polycyclic moiety optionally substituted with 1-6 C₁₋₈ alkyl groups, 1-8 halogen atoms, or a combination thereof,
R is a C₆₋₂₀ aromatic hydrocarbon group or a halogenated derivative thereof, a straight or branched chain C₂₋₂₀ alkylene group or a halogenated derivative thereof, or a C₃₋₈ cycloalkylene group or halogenated derivative thereof, and
wherein the polyetherimide comprises less than 20,000 ppm of repeating units of formula (VIII-b), repeating units of formula (VIII-c), or a combination thereof.

14. An article comprising the polyetherimide of claim 13, wherein the article is in the form of a fiber, a film, a sheet, a foam, a filament, a molded article, an extruded article, or a powder.

15. A method of manufacturing the article of claim 14 comprises molding, casting, or extruding the composition to provide the article.
